(19) United States
(12) Patent Application Publication
(10) Pub. No.: US 2022/0296511 A1
(43) Pub. Date: Sep. 22, 2022

(54) COMPOSITIONS FOR DELIVERY OF DRUG COMBINATIONS TO TREAT LUNG DISEASE

(71) Applicant: BOARD OF REGENTS, THE UNIVERSITY OF TEXAS SYSTEM, Austin, TX (US)

(72) Inventors: Robert O. WILLIAMS III, Austin, TX (US); Jay I. PETERS, San Antonio, TX (US); Tuangrat PRAPHAWATVET, Austin, TX (US); Sawittree SAHAKIJPIJARN, Austin, TX (US); Chaeho MOON, Austin, TX (US)

(73) Assignee: BOARD OF REGENTS, THE UNIVERSITY OF TEXAS SYSTEM, Austin, TX (US)

(21) Appl. No.: 17/698,906

(22) Filed: Mar. 18, 2022

Related U.S. Application Data
(60) Provisional application No. 63/162,835, filed on Mar. 18, 2021.

(51) Int. Cl. A61K 9/00; A61K 31/496; A61K 31/4412; A61K 31/365; A61K 47/10; A61K 47/12; A61K 47/18; A61K 47/32; A61K 47/40

(52) U.S. Cl. CPC A61K 9/0075; A61K 31/496; A61K 31/4412; A61K 31/365; A61K 47/10; A61K 47/12; A61K 47/183; A61K 47/32; A61K 47/40

(57) ABSTRACT

In some aspects, the present disclosure provides pharmaceutical compositions comprising particles, wherein individual particles of the composition comprise a combination of two or more active pharmaceutical ingredients selected from:
(A) nintedanib;
(B) pirfenidone; and/or
(C) mycophenolic acid.
   These compositions may be formulated for administration via inhalation. In some aspects, the present disclosure provides methods for preparing the pharmaceutical compositions of the present disclosure and methods of treating or preventing a disease or disorder using the pharmaceutical compositions of the present disclosure.

This application claims the benefit of priority to U.S. Provisional Application No. 63/162,835, filed on Mar. 18, 2021, the entire contents of which are hereby incorporated by reference.

### BACKGROUND

### 1. Field

The present disclosure relates generally to the field of pharmaceuticals and pharmaceutical manufacture. More particularly, it concerns compositions and methods of preparing a pharmaceutical composition comprising particles, wherein the individual particles of the composition comprise a combination of two or more active pharmaceutical ingredients.

### 2. Description of Related Art

Pirfenidone and nintedanib were effective and approved drugs for idiopathic pulmonary fibrosis (IPF). These two drugs delay the progression of IPF disease. Besides, mycophenolate mofetil, which is a prodrug of mycophenolic acid (MPA), decreases forced vital capacity (FVC) reduction, increases FVC capacity, and improves overall survival. The combination of mycophenolic acid and approved antifibrosis drugs especially nintedanib or pirfenidone shows more benefits for IPF patients. Oral administration of nintedanib has very low bioavailability only at 4.7%, thus this drug should be taken with food to increase absorption. Moreover, a high dose of nintedanib can increase undesirable adverse including diarrhea, nausea and vomiting, abdominal pain, decreased appetite, weight loss and hepatic enzyme increasing. Oral pirfenidone has high oral doses can cause undesirable side effects especially nausea, rash, diarrhea, fatigue, dyspepsia, anorexia, headache, and photosensitivity reaction. This drug has a narrow therapeutic index; thus, patients have to be monitored serum concentration during a treatment. Moreover, 50% of pirfenidone content can be decreased by food. Mycophenolic acid presents 40% of the maximum plasma concentration decreased by food. Furthermore, high oral doses also cause common and serious adverse drug reactions including diarrhea, leucopenia, sepsis and vomiting. In an effective treatment, two drugs were combined to treat IPF. The combinations of these drugs have a high oral dose to achieve therapeutic effects, thus patients have to tolerate adverse drug reactions. Moreover, the combination of these oral drugs has difficult to manage because nintedanib should be taken with food while pirfenidone and mycophenolic acid can be decreased by food. However, three drug combinations, which may have more benefit for IPF patients, have not been established. Inhaled single drug and two/three drug combinations may exhibit improved bioavailability and provide the therapeutic effects at a lower dose that can decrease undesirable side effects. Since idiopathic pulmonary fibrosis usually starts in the peripheral areas of the lung and spreads to more central areas of the lung, delivery of antifibrotic combination therapies to peripheral regions has clear advantages over oral therapy. As such, there is a need for pharmaceutical compositions comprising nintedanib, pirfenidone, and/or mycophenolic acid for inhalation.

### SUMMARY OF THE INVENTION

In some aspects, the present disclosure provides pharmaceutical composition comprising particles, wherein individual particles of the composition comprise a combination of two or more active pharmaceutical ingredients selected from:

(A) nintedanib;

(B) pirfenidone; and/or

(C) mycophenolic acid.

In some embodiments, the pharmaceutical compositions are formulated for administration via inhalation. In some embodiments, the particles comprise nintedanib and pirfenidone. In some embodiments, the particles comprise nintedanib and mycophenolic acid. In some embodiments, the particles comprise nintedanib, pirfenidone, and mycophenolic acid.

In some embodiments, the particles further comprise an excipient. In some embodiments, the excipient is a sugar or sugar alcohol. In some embodiments, the excipient is a sugar such as lactose, sucrose, and trehalose. In other embodiments, the excipient is a sugar alcohol such as mannitol. In other embodiments, the excipient is an acid. In some embodiments, the acid is a carboxylic acid such as fumaric acid. In other embodiments, the excipient is a cyclodextrin such as a β-cyclodextrin. In some embodiments, the excipient is a sulfo butyl ether β-cyclodextrin such as 6.5-sulfobutylether-β-cyclodextrin. In other embodiments, the excipient is an amino acid. In some embodiments, the amino acid is a hydrophobic amino acid such as leucine. In other embodiments, the excipient is a flow enhancing agent such as magnesium stearate. In other embodiments, the excipient is lecithin. In other embodiments, the excipient is a pharmaceutically acceptable polymer. In some embodiments, the pharmaceutically acceptable polymer is a non-cellulosic polymer such as a non-ionizable non-cellulosic polymer. In some embodiments, the pharmaceutical acceptable polymer is polyvinylpyrrolidone. In some embodiments, the polyvinylpyrrolidone comprises a molecular weight from about 10,000 to about 40,000. In some embodiments, the molecular weight is from about 20,000 such as about 24,000.

In some embodiments, the particles comprise from about 10% w/w to about 80% w/w of the active pharmaceutical ingredients. In some embodiments, the particles comprise from about 15% w/w to about 60% w/w of the active pharmaceutical ingredients. In some embodiments, the particles comprise from about 20% w/w to about 40% w/w of the active pharmaceutical ingredients such as about 25% w/w of the active pharmaceutical ingredients.

In some embodiments, the particles comprise a weight ratio of nintedanib and pirfenidone from about 5:1 to about 1:10. In some embodiments, the weight ratio of nintedanib and pirfenidone in the particles is from about 1:1 to about 1:5 such as about 1:3. In some embodiments, the particles comprise a weight ratio of nintedanib and mycophenolic acid from about 5:1 to about 1:10. In some embodiments, the weight ratio of nintedanib and mycophenolic acid in the particles is from about 1:1 to about 1:5 such as about 1:3. In some embodiments, the particles comprise a weight ratio of pirfenidone and mycophenolic acid from about 10:1 to about 1:10. In some embodiments, the weight ratio of pirfenidone and mycophenolic acid in the particles is from about 5:1 to about 1:5 such as about 1:1.

In some embodiments, the particles comprise from about 50% w/w to about 95% w/w of the excipient. In some embodiments, the particles comprise from about 65% w/w to about 85% w/w of the excipient such as about 75% w/w of the excipient.

In some embodiments, the particles comprise at least 80% of one or more of the active pharmaceutical ingredients in the amorphous phase. In some embodiments, at least 90% of one or more of the active pharmaceutical ingredients is in the amorphous phase. In some embodiments, at least 95% of one or more of the active pharmaceutical ingredients is in the amorphous phase. In some embodiments, at least 98% of one or more of the active pharmaceutical ingredients is in the amorphous phase. In some embodiments, at least 99% of one or more of the active pharmaceutical ingredients is in the amorphous phase.

In some embodiments, the active pharmaceutical ingredient in the amorphous form is nintedanib. In some embodiments, the active pharmaceutical ingredient in the amorphous form is pirfenidone. In some embodiments, the active pharmaceutical ingredient in the amorphous form is mycophenolic acid. In some embodiments, the active pharmaceutical ingredient in the amorphous form is nintedanib and pirfenidone. In some embodiments, the active pharmaceutical ingredient in the amorphous form is nintedanib and mycophenolic acid. In some embodiments, the active pharmaceutical ingredient in the amorphous form is nintedanib, pirfenidone, and mycophenolic acid.

In some embodiments, the particles comprise at least 80% of the excipient in the amorphous phase. In some embodiments, at least 90% of excipient is in the amorphous phase. In some embodiments, at least 95% of the excipient is in the amorphous phase. In some embodiments, at least 98% of the excipient is in the amorphous phase. In some embodiments, at least 99% of the excipient is in the amorphous phase. In other embodiments, the particles comprise at least 80% of the excipient in the crystalline phase. In some embodiments, at least 90% of excipient is in the crystalline phase. In some embodiments, at least 95% of the excipient is in the crystalline phase. In some embodiments, at least 98% of the excipient is in the crystalline phase. In some embodiments, at least 99% of the excipient is in the crystalline phase.

In some embodiments, the particles comprise a matrix structure. In some embodiments, the particles comprise a homogenous mixture of the active pharmaceutical ingredients.

In some embodiments, the particles containing nintedanib has a mass median aerodynamic diameter from about 1.0 μm to about 6.0 μm. In some embodiments, the mass median aerodynamic diameter of the particles containing nintedanib is from about 2.0 μm to about 5.0 μm such as from about 2.5 μm to about 4.5 μm.

In some embodiments, the particles containing pirfenidone has a mass median aerodynamic diameter from about 1.0 μm to about 7.0 μm. In some embodiments, the mass median aerodynamic diameter of the particles containing pirfenidone is from about 2.0 μm to about 6.0 μm such as from about 3.0 μm to about 5.0 μm. In some embodiments, the particles containing mycophenolic acid has a mass median aerodynamic diameter from about 1.0 μm to about 6.0 μm. In some embodiments, the mass median aerodynamic diameter of the particles containing mycophenolic acid is from about 1.5 μm to about 5.0 μm such as from about 2.0 μm to about 4.5 μm.

In some embodiments, the particles containing nintedanib has a geometric standard deviation (GSD) from about 1 to about 7.5. In some embodiments, the geometric standard deviation of the particles containing nintedanib is from about 1.5 to about 5 such as from about 2 to about 4. In some embodiments, the particles containing pirfenidone has a geometric standard deviation (GSD) from about 1 to about 8. In some embodiments, the geometric standard deviation of the particles containing pirfenidone is from about 1.5 to about 6.5 such as from about 2 to about 5.5. In some embodiments, the particles containing mycophenolic acid has a geometric standard deviation (GSD) from about 1 to about 7.5. In some embodiments, the geometric standard deviation of the particles containing mycophenolic acid is from about 1.5 to about 5 such as from about 2 to about 4.

In some embodiments, the pharmaceutical composition has a fine particle fraction of recovered dose of the particles containing nintedanib is greater than 15%. In some embodiments, the fine particle fraction of recovered dose of the particles containing nintedanib is greater than 20% such as greater than 25%. In some embodiments, the pharmaceutical composition has a fine particle fraction of recovered dose of the particles containing pirfenidone is greater than 15%. In some embodiments, the fine particle fraction of recovered dose of the particles containing pirfenidone is greater than 20% such as greater than 25%. In some embodiments, the pharmaceutical composition has a fine particle fraction of recovered dose of the particles containing mycophenolic acid is greater than 15%. In some embodiments, the fine particle fraction of recovered dose of the particles containing mycophenolic acid is greater than 18% such as greater than 20%.

In some embodiments, the pharmaceutical composition has a fine particle fraction of delivered dose of the particles containing nintedanib is greater than 25%. In some embodiments, the fine particle fraction of delivered dose of the particles containing nintedanib is greater than 30% such as greater than 35%. In some embodiments, the pharmaceutical composition has a fine particle fraction of delivered dose of the particles containing pirfenidone is greater than 20%. In some embodiments, the fine particle fraction of delivered dose of the particles containing pirfenidone is greater than 25 such as greater than 30%. In some embodiments, the pharmaceutical composition has a fine particle fraction of delivered dose of the particles containing mycophenolic acid is greater than 20%. In some embodiments, the fine particle fraction of delivered dose of the particles containing mycophenolic acid is greater than 25% such as greater than 30%.

In some embodiments, the pharmaceutical composition has a percentage recovery as a function of the loaded dose of the particles containing nintedanib is greater than 60%. In some embodiments, the percentage recovery as a function of the loaded dose of the particles containing nintedanib is greater than 65% such as greater than 70%. In some embodiments, the pharmaceutical composition has a percentage recovery as a function of the loaded dose of the particles containing pirfenidone is greater than 60%. In some embodiments, the percentage recovery as a function of the loaded dose of the particles containing pirfenidone is greater than 65% such as greater than 70%. In some embodiments, the pharmaceutical composition has a percentage recovery as a function of the loaded dose of the particles containing mycophenolic acid is greater than 70%. In some embodiments, the percentage recovery of the loaded dose as a function of the particles containing mycophenolic acid is greater than 75% such as greater than 80%.

In some embodiments, the pharmaceutical composition has an emitted fraction of the particles containing nintedanib is greater than 60% as measured using a NGI. In some embodiments, the emitted fraction of the particles containing nintedanib is greater than 65% such as greater than 70%. In some embodiments, the pharmaceutical composition has an emitted fraction of the particles containing pirfenidone is greater than 60% as measured using a NGI. In some embodiments, the emitted fraction of the particles containing pirfenidone is greater than 65% such as greater than 70%. In some embodiments, the pharmaceutical composition has an emitted fraction of the particles containing mycophenolic acid is greater than 70% as measured using a NGI. In some embodiments, the emitted fraction of the particles containing mycophenolic acid is greater than 75% such as greater than 80%.

In another aspect, the present disclosure provides pharmaceutical compositions comprising particles, wherein individual particles of the composition comprise a combination of an active pharmaceutical ingredient and an excipient comprising:
(A) the active pharmaceutical ingredient selected from nintedanib, pirfinedone, and mycophenolic acid;
(B) the excipient;
wherein the pharmaceutical composition is formulated as a dry powder for administration via inhalation.

In some embodiments, the active pharmaceutical ingredient is nintedanib. In some embodiments, the active pharmaceutical ingredient is pirfinedone. In some embodiments, the active pharmaceutical ingredient is mycophenolic acid.

In some embodiments, the particles further comprise an excipient. In some embodiments, the excipient is a sugar or sugar alcohol. In some embodiments, the excipient is a sugar such as lactose. In other embodiments, the excipient is a sugar alcohol such as mannitol. In other embodiments, the excipient is a cyclodextrin such as a β-cyclodextrin. In some embodiments, the excipient is a sulfo butyl ether β-cyclodextrin such as 6.5-sulfobutylether-β-cyclodextrin. In other embodiments, the excipient is an amino acid. In some embodiments, the amino acid is a hydrophobic amino acid such as leucine. In other embodiments, the excipient is a flow enhancing agent such as magnesium stearate. In other embodiments, the excipient is lecithin. In other embodiments, the excipient is a pharmaceutically acceptable polymer. In some embodiments, the pharmaceutically acceptable polymer is a non-cellulosic polymer such as a non-ionizable non-cellulosic polymer. In some embodiments, the pharmaceutical acceptable polymer is polyvinylpyrrolidone. In some embodiments, the polyvinylpyrrolidone comprises a molecular weight from about 10,000 to about 40,000. In some embodiments, the molecular weight is from about 20,000 such as about 24,000.

In some embodiments, the particles comprise from about 10% w/w to about 80% w/w of the active pharmaceutical ingredients. In some embodiments, the particles comprise from about 15% w/w to about 60% w/w of the active pharmaceutical ingredients. In some embodiments, the particles comprise from about 20% w/w to about 40% w/w of the active pharmaceutical ingredients such as about 25% w/w of the active pharmaceutical ingredients. In other embodiments, the particles comprise from about 1% w/w to about 40% w/w of the active pharmaceutical ingredients. In some embodiments, the particles comprise from about 5% w/w to about 20% w/w of the active pharmaceutical ingredients. In some embodiments, the particles comprise from about 7.5% w/w to about 17.5% w/w of the active pharmaceutical ingredients. In other embodiments, the particles comprise about 10% w/w of the active pharmaceutical ingredients. In other embodiments, the particles comprise about 15% w/w of the active pharmaceutical ingredients. In some embodiments, the particles comprise from about 50% w/w to about 95% w/w of the excipient. In some embodiments, the particles comprise from about 65% w/w to about 85% w/w of the excipient such as about 75% w/w of the excipient. In other embodiments, the particles comprise from about 75% w/w to about 95% w/w of the excipient. In some embodiments, the particles comprise about 90% w/w of the excipient. In other embodiments, the particles comprise about 85% w/w of the excipient.

In some embodiments, the particles comprise at least 80% of one or more of the active pharmaceutical ingredients in the amorphous phase. In some embodiments, at least 90% of one or more of the active pharmaceutical ingredients is in the amorphous phase. In some embodiments, at least 95% of one or more of the active pharmaceutical ingredients is in the amorphous phase. In some embodiments, at least 98% of one or more of the active pharmaceutical ingredients is in the amorphous phase. In some embodiments, at least 99% of one or more of the active pharmaceutical ingredients is in the amorphous phase.

In some embodiments, the particles comprise at least 80% of the excipient in the amorphous phase. In some embodiments, at least 90% of excipient is in the amorphous phase. In some embodiments, at least 95% of the excipient is in the amorphous phase. In some embodiments, at least 98% of the excipient is in the amorphous phase. In some embodiments, at least 99% of the excipient is in the amorphous phase. In other embodiments, the particles comprise at least 80% of the excipient in the crystalline phase. In some embodiments, at least 90% of excipient is in the crystalline phase. In some embodiments, at least 95% of the excipient is in the crystalline phase. In some embodiments, at least 98% of the excipient is in the crystalline phase. In some embodiments, at least 99% of the excipient is in the crystalline phase.

In another embodiment, the present disclosure provides methods of preparing a pharmaceutical composition described herein comprising:
(A) dissolving an active pharmaceutical ingredient in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a pharmaceutical composition.

In some embodiments, the solvent is an organic solvent. In some embodiments, the solvent is acetonitrile, tert-butanol, or 1,4-dioxane. In some embodiments, the methods further comprise admixing the active pharmaceutical ingredient with an excipient. In some embodiments, the pharmaceutical mixture further comprises a second solvent such as water. In some embodiments, the solvent is mixed with the second solvent to obtain a homogenous pharmaceutical mixture. In some embodiments, the pharmaceutical mixture is admixed until the pharmaceutical mixture is clear.

In some embodiments, the pharmaceutical mixture comprises a solid content from about 0.05% w/v to about 5% w/v of the active pharmaceutical ingredient and the excipient. In some embodiments, the solid content is from about 0.1% w/v to about 2.5% w/v of the active pharmaceutical ingredient and the excipient. In some embodiments, the solid content is from about 0.15% w/v to about 1.5% w/v of the active pharmaceutical ingredient and the excipient. In some embodiments, the solid content is from about 0.2% w/v to about 0.6% w/v of the active pharmaceutical ingredient and the excipient. In some embodiments, the solid content is from about 0.5% w/v to about 1.25% w/v of the active pharmaceutical ingredient and the excipient.

In some embodiments, the pharmaceutical mixture is applied at a feed rate from about 0.5 mL/min to about 5 mL/min. In some embodiments, the feed rate is from about 1 mL/min to about 3 mL/min such as about 2 mL/min. In some embodiments, the pharmaceutical mixture is applied with a nozzle such as a needle. In some embodiments, the pharmaceutical mixture is applied from a height from about 2 cm to about 50 cm. In some embodiments, the height is from about 5 cm to about 20 cm such as about 10 cm.

In some embodiments, the surface temperature is from about 0° C. to −190° C. In some embodiments, the surface temperature is from about −25° C. to about −125° C. such as about −100° C. In some embodiments, the surface is a rotating surface. In some embodiments, the surface is rotating at a speed from about 5 rpm to about 500 rpm. In some embodiments, the surface is rotating at a speed from about 100 rpm to about 400 rpm such as a speed of about 200 rpm.

In some embodiments, the frozen pharmaceutical composition is dried by lyophilization. In some embodiments, the frozen pharmaceutical composition is dried at a first reduced pressure. In some embodiments, the first reduced pressure is from about 10 mTorr to 500 mTorr. In some embodiments, the first reduced pressure is from about 50 mTorr to about 250 mTorr such as about 100 mTorr. In some embodiments, the frozen pharmaceutical composition is dried at a first reduced temperature. In some embodiments, the first reduced temperature is from about 0° C. to −100° C. In some embodiments, the first reduced temperature is from about −20° C. to about −60° C. such as about −40° C. In some embodiments, the frozen pharmaceutical composition is dried for a primary drying time period from about 3 hours to about 36 hours. In some embodiments, the primary drying time period is from about 6 hours to about 24 hours such as about 20 hours.

In some embodiments, the frozen pharmaceutical composition is dried a secondary drying time period. In some embodiments, the frozen pharmaceutical composition is dried a secondary drying time at a second reduced pressure. In some embodiments, the secondary drying time is at a reduced pressure is from about 10 mTorr to 500 mTorr. In some embodiments, the secondary drying time is at a reduced pressure is from about 50 mTorr to about 250 mTorr such as about 100 mTorr. In some embodiments, the frozen pharmaceutical composition is dried a secondary drying time at a second reduced temperature. In some embodiments, the second reduced temperature is from about 0° C. to 30° C. In some embodiments, the second reduced temperature is from about 10° C. to about 30° C. such as about 25° C. In some embodiments, the frozen pharmaceutical composition is dried for a second time for a second time period from about 3 hours to about 36 hours. In some embodiments, the second time period is from about 6 hours to about 24 hours such as about 20 hours. In some embodiments, the temperature is changed from the first reduced temperature to the second reduced temperature over a ramping time period. In some embodiments, the ramping time period is from about 3 hours to about 36 hours. In some embodiments, the ramping time period is from about 6 hours to about 24 hours such as about 20 hours.

In another aspect, the present disclosure provides pharmaceutical compositions prepared using the methods described herein.

In still another aspect, the present disclosure provides methods of treating or preventing a lung disease in a patient comprising administering to the patient in need thereof a therapeutically effective amount of a pharmaceutical composition described herein. In some embodiments, the lung disease is associated with lung inflammation or fibrosis. In some embodiments, the lung disease is interstitial lung disease such as idiopathic pulmonary fibrosis. In some embodiments, the weight ratio of nintedanib to pirfenidone is from about 1:1 to about 1:10. In some embodiments, the weight ratio is from about 1:2 to about 1:5 such as about 1:3. In some embodiments, the weight ratio of nintedanib to mycophenolic acid is from about 1:1 to about 1:10. In some embodiments, the weight ratio is from about 1:2 to about 1:5 such as about 1:3.

In some embodiments, the pharmaceutical composition comprises a dose of nintedanib is from about 1 mg/mL to about 50 mg/mL. In some embodiments, the dose of nintedanib is from about 2.5 mg/mL to about 25 mg/mL such as from about 5 mg/mL to about 20 mg/mL. In some embodiments, the pharmaceutical composition comprises a dose of pirfenidone is from about 0.25 mg to about 10 mg. In some embodiments, the dose of pirfenidone is from about 0.5 mg to about 7.5 mg such as from about 0.75 mg to about 5 mg. In some embodiments, the pharmaceutical composition comprises a dose of mycophenolic acid is from about 0.25 μg/mL to about 10 μg/mL. In some embodiments, the dose of mycophenolic acid is from about 0.5 μg/mL to about 7.5 μg/mL such as from about 0.75 μg/mL to about 5 μg/mL.

In still yet another aspect, the present disclosure provides methods of treating or prevent reducing lung inflammation or fibrosis in a patient comprising administering to the patient in need thereof a therapeutically effective amount of a pharmaceutical composition described herein. In some embodiments, the lung inflammation or fibrosis is associated with an interstitial lung disease. In some embodiments, the interstitial lung disease is idiopathic pulmonary fibrosis. In some embodiments, the pharmaceutical composition is administered once. In other embodiments, the pharmaceutical composition is administered more than once.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1C shows XRPD of δ-mannitol, three-drug combination of nintedanib, pirfenidone, mycophenolic acid (T01, T02, T03, T04; FIG. 1A); two-drug combinations of nintedanib and pirfenidone (F06, F07, F08, F09; FIG. 1B); two-drug combinations of nintedanib and mycophenolic acid (NM01, NM02, NM03, NM04; FIG. 1C).

FIG. 2 shows morphology of triple-drug combination formulations at different magnifications (1.00 K×, 3.00 K×, 5.00 K×, 10.00 K×)

FIG. 3 shows morphology of two-drug combination formulations (nintedanib and pirfenidone) at different magnifications (1.00 K×, 3.00 K×, 5.00 K×, 10.00 K×).

FIG. 4 shows morphology of two-drug combination formulations (nintedanib and mycophenolic acid) at different magnifications (1.00 K×, 3.00 K×, 5.00 K×, 10.00 K×).

FIG. 5 shows XRPD of δ-mannitol, three-drug combination of nintedanib, pirfenidone, mycophenolic acid (T01, T02,); two-drug combination of nintedanib and pirfenidone (F06); two-drug combination of nintedanib and mycophenolic acid (NM02).

FIG. 6 shows morphology of TFF powders for inhalations at different magnifications (3.00 K×, 5.00 K×, 10.00 K×).

FIG. 7 shows morphology of triple-drug combination formulations at different magnifications (1.00 K×, 5.00 K×, 10.00 K×, 20.0 K×).

FIG. 8 shows T01 drug deposition (%) of each stage from NGI.

FIG. 9 shows T02 drug deposition (%) of each stage from NGI.

FIG. 10 shows T01_L25 drug deposition (%) of each stage from NGI.

FIG. 11 shows T02_L25 drug deposition (%) of each stage from NGI.

FIG. 12 shows the powder X-ray diffraction of nintedanib compositions compared to neat leucine, mannitol, and nintedanib. The graph shows the change in these compositions over 3 months showing no significant change in crystallization.

FIG. 13 shows the powder X-ray diffraction of nintedanib after storage at 40° C. for 2 weeks. These compositions show little change in crystallization over that time.

FIG. 14 shows the fine powder fraction (recovered), fine powder fraction (delivered), MMAD, and % recovery along with SEM for 2 formulations of nintedanib after preparation, then after 1 and 3 minutes at room temperature.

FIG. 15 shows the fine powder fraction (recovered), fine powder fraction (delivered), MMAD, and % recovery along with SEM for 4 formulations of nintedanib after preparation and after 2 weeks of storage at 40° C.

FIG. 16 shows the distribution of particles into the respiratory system after delivery through an inhaler for compositions N03 and N04.

FIG. 17 shows the distribution of particles into the respiratory system after delivery through an inhaler for composition N14, N15, N17, and N18.

FIG. 18 shows the powder X-ray diffraction of nintedanib, pirfenidone, and mycophenolic acid compositions (T10, T11, T35, T36, T37, T38, and T40) compared to the individual ingredients.

FIG. 19 shows the powder X-ray diffraction of nintedanib and mycophenolic acid compositions (NM08 and NM 09) compared to the individual ingredients.

FIG. 20 shows the SEM of nintedanib, pirfenidone, and mycophenolic acid compositions (T10, T11, T35, T36, T37, T38, and T40) at 3×, 5×, and 10×.

FIG. 21 shows the SEM of nintedanib and mycophenolic acid compositions (NM08 and NM 09) at 3×, 5×, and 10×.

FIG. 22 shows the distribution into the respiratory system after inhalation of nintedanib, pirfenidone, and mycophenolic acid compositions (T10, T11, T35, T36, T37, and T38)

FIG. 23 shows the distribution into the respiratory system after inhalation of nintedanib, pirfenidone, and mycophenolic acid compositions (T40)

FIG. 24 shows the distribution into the respiratory system after inhalation of nintedanib and mycophenolic acid compositions (NM08 and NM 09).

FIG. 25 shows the powder X-ray diffraction spectrum of pirfenidone compositions (P9, P17, P18, P20, P21, and P22) compared to pirfenidone.

FIG. 26 shows the powder X-ray diffraction spectrum of pirfenidone compositions (P23, P24, P25, P26, and P27) compared to pirfenidone and leucine.

FIG. 27 shows the SEM of pirfenidone compositions (P9, P17, P18, P20, P21, P22, and P23).

FIG. 28 shows the SEM of pirfenidone compositions (P24, P25, P26, and P27)

FIG. 29 shows the distribution into the respiratory system after inhalation of pirfenidone compositions (P9, P17, P18, P20, P21, P22, and P23).

FIG. 30 shows the distribution into the respiratory system after inhalation of pirfenidone compositions (P24, P25, P26, and P27).

### DESCRIPTION OF ILLUSTRATIVE EMBODIMENTS

In some aspects, the present disclosure relates to pharmaceutical compositions comprising composite particles containing nintedanib, pirfenidone, and/or mycophenolic acid capable of being delivered to the upper and lower airways in the treatment of lung diseases, including those associated with lung inflammation or fibrosis, such as interstitial lung disease and idiopathic pulmonary fibrosis. The composite particles are engineered in such a way that the resulting composition may be delivered in powder form using a dry powder inhaler (DPI) to the lower airways. The ability to deliver the pharmaceutical compositions using a range of delivery systems without the need for changes to the powder components and ratios or processing methods makes the composition broadly applicable to a range of patient populations, and includes those who are ambulatory or in an out-patient setting, patients with reduced lung function or those who may require mechanical ventilation, and pediatric or geriatric who may exhibit reduced inspiratory capacity. Also provided herein are methods of preparing and using these compositions. Details of these compositions are provided in more detail below.

### I. PHARMACEUTICAL COMPOSITIONS

In some aspects, the present disclosure provides pharmaceutical compositions comprising composite particles containing nintedanib, pirfenidone, and/or mycophenolic acid that may be formulated for administration to the lungs.

A. Inhaled Drug Combinations for Pulmonary Fibrosis

Nintedanib and pirfenidone have been separately administered to treat pulmonary fibrosis especially idiopathic pulmonary fibrosis (IPF). Recently, the combination of nintedanib and pirfenidone has been encouraged in the treatment of IPF patients because this combination has manageable safety and tolerability profile. Moreover, mycophenolic acid combined with other antifibrotic drugs was found beneficial to treat IPF. In the literature, oral delivery of two-drug combinations including nintedanib, pirfenidone or mycophenolic acid is effective for pulmonary fibrosis. However, three-drug combination, which is possible to provide more effective for IPF treatment, has not been reported to treat pulmonary fibrosis.

Furthermore, the use of these drugs as oral formulations suffers from significant disadvantages as described for each individual drugs below. These disadvantages include significant side effects and narrow useful therapeutic windows. For example, oral administrations of nintedanib suffer from significant side effects such as nausea and low oral bioavailability. Pirfenidone, on the other hand, is associated with severe side effects due to the high doses that must be given. Furthermore, formulation of these drugs as an oral combination presents unique challenges because nintedanib should be taken with food while the absorbance and activity of pirfenidone and mycophenolic acid is reduced by food. Furthermore, individual administration through inhalation is plagued by different delivery of the therapeutic agent as well as patient compliance concerns. For this reason, the development of a system capable of delivering a combined dose of these drugs may be particularly useful in treating interstitial lung disease.

B. Interstitial lung disease (ILD) & Treatment

Interstitial lung disease (ILD) is a term describing diseases that occur in the tissues and spaces between alveolus. To classify interstitial disease, respiratory symptoms, pulmonary function, and inflammation and fibrosis were investigated (Schraufnagel, 2010). Thus, over 200 lung diseases can be identified as ILD, such as idiopathic pulmonary fibrosis (IPF), hypersensitivity pneumonitis, sarcoidosis, and asbestosis (Schraufnagel, 2010; American Lung Association, 2020). ILD especially IPF often occurs in adults at the ages of 40 and 70 (Schraufnagel, 2010). The common cause of ILD is idiopathic condition; however, IPD can be caused by other diseases associated with lung damage, immune reaction, and genetic abnormalities (American Thoracic Society/European Respiratory Society, 2002). The treatment of ILD often begins with medications including corticosteroids and immunosuppressive agents to decrease the inflammation of the connective tissue related lung disease (Demedts et al., 2005). Idiopathic pulmonary fibrosis (IPF) is a type of idiopathic ILD that is classified as chronic and progressive pulmonary fibrosis (Hickey and Mansour, 2019). In IPF patients who have severe symptoms, lung transplantation is an effective treatment that can increase survival. However, over 30 percentage of patients die before receiving lung transplantation, and only 40 percentage of post-transplant patients have five-year survival (Trulock et al., 2007). As the results, medications that can postpone the progression of lung disease and improve lung functions appear as the effective treatment for IPF patients (Trulock et al., 2007).

In 2014, pirfenidone (Esbriet®) and nintedanib (Ofev®) were the first FDA-approved drugs for IPF treatment. These two drugs can delay disease progression for mild to moderate IPF patients (Hickey and Mansour, 2019). Mycophenolate mofetil (CellCept®), which is a prodrug of mycophenolic acid (MPA), can decrease forced vital capacity (FVC) reduction, increase FVC capacity, and improve overall survival compared with ineffective/harmful treatment or no specific treatment (Nambiar et al., 2017).

i. Nintedanib

Nintedanib (Ofev®) was approved for the treatment of idiopathic pulmonary fibrosis (IPF) in adults. The recommended close is 150 mg twice a day administered approximately every 12 hours (Summary of Product Characteristics: Ofev, 2019). Nintedanib has anti-fibrosis activity because of mechanisms including tyrosine kinase inhibitor and inhibiting of the adenosine triphosphate (ATP) binding receptors to block intracellular signaling (Summary of Product Characteristics: Ofev, 2019). This drug can inhibit tyrosine kinase via platelet-derived growth factor receptor (PDGFR) α and β, fibroblast growth factor receptor (FGFR) 1-3, and VEGFR 1-3. Moreover, nintedanib can also have the ability to inhibit Flt-3 (Fms-like tyrosine-protein kinase), Lck (lymphocyte-specific tyrosine-protein kinase), Lyn (tyrosine-protein kinase Lyn) and Src (proto-oncogene tyrosine-protein kinase Src) kinases (Summary of Product Characteristics: Ofev, 2019).

In a study, 150 mg nintedanib twice daily can reduce the decrease of FVC and delay the disease progression in IPF patients who were at the age of 40 years or older. However, five percentage of patients had to be excluded from the study because of diarrhea that was an adverse drug reaction of this drug (Richeldi et al., 2014). In phase 3 trial, nintedanib decreased the annual rate of FVC reduction and slow rate of ILD progression compared with placebo. In a nintedanib group, the reduction of FVC was −80.0 mL per year and 187.8 mL per year in a placebo group (Flaherty et al 2019).

On the other hand, oral administration of nintedanib has very low oral bioavailability at 4.7% and causes undesirable adverse reactions that were observed in researches and post-marketing period. In phase 3 trial, the most common adverse reaction was diarrhea that was reported in 66.9%; moreover, nausea and vomiting and abnormality of the test of liver-function were found in the nintedanib group more than the placebo group (Summary of Product Characteristics: Ofev, 2019; Flaherty et al., 2019). Adverse reactions including diarrhea, nausea and vomiting, abdominal pain, decreased appetite, weight decreased and hepatic enzyme increased were mostly reported in the post-marketing period (Summary of Product Characteristics: Ofev, 2019).

Pulmonary delivery of nintedanib was studied for ILD treatment. In clinical trials, twice daily of the aerosol solution containing nintedanib and olodaterol had effective to treat ILD. Liquid formulations of nintedanib monoethanesulphonate and olodaterol hydrochloride were prepared to administer for clinical studied. Moreover, a dry powder formulation for inhalation containing nintedanib monoethanesulphonate, mannitol and L-leucine nanocrystals coating was produced by aerosol flow reactor method to obtain flow/able and dispersible powders. Besides, the inhaled powder combinations of nintedanib monoethanesulphonate and olodaterol hydrochloride were also developed for ILD treatment (U.S. Pat. No. 9,517,204).

As used herein, nintedanib refers to the free base compound, a salt, a crystal form, a co-crystal, or a pro-drug thereof. In particular, nintedanib may be the salt form of the nintedanib such as nintedanib esylate.

ii. Pirfenidone

Pirfenidone (Esbriet®), known as an immunosuppressant, is approved for mild to moderate IPF in adults. Patients have to take a high daily dose to reach the therapeutic range; thus, the recommended daily doses are 801 mg in the first week, 1602 mg in the second week, and 2403 mg in the third week onward (Summary of Product. Characteristics: Esbriet, 2015). This drug has complicated management because 50% of pirfenidone can be decreased by food; moreover, a motoring serum concentration is necessary for patients because of the narrow therapeutic index (Summary of Product Characteristics: Esbriet, 2015).

In in vitro and in vivo studies, pirfenidone showed antifibrotic and anti-inflammatory properties that reduce inflammatory cells aggregation (Summary of Product Characteristics: Esbriet, 2015). Pirfenidone can decrease proinflammatory cytokines including tumor necrosis factor-alpha (TNF-α), interferon γ (IFNγ), and interleukin (IL)-6; moreover, this drug showed an antioxidant property to prevent cells hydroxyl superoxide anion free radicals (Margaritopoulos et al., 2016). Pirfenidone can also reduce platelet-derived growth factor (PDGF) and COL1A1 expression that involves the mechanism of pulmonary fibrosis (Lopez-de la Mora et al., 2015). In addition, fibrocyte accumulation, cell migration and proliferation were inhibited by pirfenidone via protein-coupled receptors including CCl12, CCR2, and GLI transcription factors (Inomata et al., 2014; Didiasova et al., 2017). On account of the high oral dose, patients have to tolerate with adverse drug reactions including nausea, rash, diarrhea, fatigue, dyspepsia, anorexia, headache, and photosensitivity reaction (Summary of Product Characteristics: Esbriet, 2015).

Recently, pirfenidone for inhalation was studied. In phase 1 study of aerosolized pirfenidone, 12.5 mg/ml pirfenidone was prepared and delivered by the eFlow investigational vibrating mesh nebulizer (PARI, Germany). Normal volunteers received 25, 50, 100 mg, and IPF patients received 100 mg. Pharmacokinetic data presented inhaled pirfenidone can improve lung concentration of pirfenidone compared with effective oral doses. Aerosol epithelial lining fluid (ELF) concentrations of 100 mg aerosol pirfenidone was 35 times more than 801 mg. Furthermore, adverse effects profile showed the higher dose was received, the more adverse reactions can occur. As the results, a 100 mg inhalation dose, which was delivered to the systemic circulation around 40-45 mg, may provide preferable safety profiles because inhaled pirfenidone existed 15-fold less systemic exposure than the oral delivery (Khoo et al., 2020).

Moreover, inhaled powders of pirfenidone can reduce risk of photodermatosis that is a critical side effect of this drug. Micronized powders of pirfenidone were produced by a grinder such as a jet mill to be obtained diameter of 20 μm that can deliver to lungs aerodynamically. The micronized powders were mixed with lactose which is a saccharide carrier and has a mean particle diameter of 10 to 100 μm. The subjects, who were received a therapeutic dose of inhaled powders at 0.1 mg/kg, significantly receive the reduction of skin extraction rate. Therefore, dry powder inhaler formulation can decrease a drug-induced photodermatosis risk (PCT Publication No. WO 2018/108669).

As used herein, pirfenidone refers to the free base compound, a salt, a crystal form, a co-crystal, or a pro-drug thereof.

iii. Mycophenolic Acid

Mycophenolic acid, which has antifibrotic and immunosuppressant activities, can be used to treat pulmonary fibrosis and prevent allograft rejection (Morath et al., 2006). Mycophenolic acid appears as highly selective, uncompetitive and reversible inhibitor of inosine monophosphate dehydrogenase (IMPDH) involving in purine nucleotide synthesis. Thus, this drug can limit the proliferation of T- and B-lymphocytes, monocytes and macrophages (Morath et al., 2006; Fujiyama et al., 2009; Jonsson and Carlsten, 2002). Moreover, mycophenolic acid reduces various cytokines including IFN-γ in macrophage and TGF-β1 in profibrotic process (Morath et al., 2006; Jonsson and Carlsten, 2002).

In vitro and animal studies confirmed mycophenolic acid has antifibrotic and antiproliferative properties on non-immune cells. The antifibrotic property on various cells such as lung fibroblasts (human), fibroblasts (rat), and tenon fibroblasts (human) were showed in the in vivo studies. In animal models, mycophenolic acid also showed antifibrotic and antiproliferative properties on rat models (PCT Publication No. WO 2018/108669).

Recently, mycophenolate is suggested as first line therapy of pulmonary fibrosis associated with scleroderma (systemic sclerosis). Mycophenolic acid is available to treat IPF by declining the IPF progression. In IPF patients, mycophenolic acid can decrease the reduction of forced vital capacity (FVC), improve FVC stability, and increase overall survival compared with ineffective/harmful treatment with prednisone, azathioprine, and/or N-acetyl cysteine and no specific treatment (Nambiar et al., 2017). Furthermore, mycophenolic acid shows the ability to maintain IPF progression, preferable adverse reaction profile, and lower cost of the treatment compared with other antifibrosis drugs. In a practical treatment, the combination of mycophenolic acid and approved antifibrosis drugs can provide benefits for IPF patients (Nambiar et al., 2017). Mycophenolate mofetil (Cellcept®), which is a prodrug of mycophenolic acid, was studied in rats to compare the pharmacokinetic profile and systemic bioavailability of oral and pulmonary delivery. Mycophenolate mofetil was prepared in suspension for aerosol inhalation via nebulizer. The study presented mycophenolate mofetil suspension for the pulmonary delivery provided high and maintained the concentration of drug in lung and lymphatic system; however, plasma concentration appeared at lower levels compared with the oral delivery of mycophenolate mofetil (Cellcept®; Dugas et al., 2013).

As used herein, mycophenolic acid refers to the free base compound, a salt, an ester, a crystal form, a co-crystal, or a pro-drug thereof. In particular, the mycophenolic acid may be mycophenolic acid, the sodium salt of mycophenolic acid, mycophenolate sodium, or the morpholino ester pro-drug, mycophenolate mofetil.

F. Thin-Film Freezing Brittle Matrix Powder Combinations for IPF

The 2015 clinical guidelines for idiopathic pulmonary fibrosis, which was published by American Thoracic Society (ATS)/European Respiratory Society (ERS)/Japanese Respiratory Society (JRS)/Latin American Thoracic Society (ALAT), recommended treating IPF patients with nintedanib and pirfenidone in moderate confidence in effect. In clinical use, nintedanib and pirfenidone reduce the progression of IPF disease because these two drugs decrease the reduction rate of FCV and improve clinical outcomes such as survival and acute exacerbations (Raghu et al., 2015; Wilson and Raghu, 2015). The safety profile was asserted to support the combination of nintedanib and pirfenidone for IPF patients. Treatment-emergent adverse events (TEAEs) was investigated in IPF patients who received a daily dose of 200-300 mg nintedanib and 1,602-2,403 pirfenidone for 24 weeks. Most of IPF patients had drugs toleration and similar kinds of TEAEs compared with monotherapy (Flaherty et al., 2018).

In terms of inhaled delivery, pirfenidone and mycophenolic acid, these drugs possess benefit in the treatment of IPF (Khoo et al., 2020; Dugas et al., 2013). Aerosolized pirfenidone that was delivered via nebulizer exhibited high ELF concentration and lower systemic exposure compared with the oral delivery (Khoo et al., 2020). IV dose 40 mg/kg showed lung tissue concentration was nine percentage of plasma concentration. The plasma concentration of 801 mg oral dose was presented at 5 μg/mL; thus, lung concentration should have 0.7 μg/g. As a result, an inhaled delivery dose of pirfenidone should be 3,733 μg, when 30% respirable delivered close (RDD) was assumed (PCT Publication No. WO 2012/106382). Moreover, the therapeutic range of mycophenolic acid is 1.0-3.5 mcg/ml (Hiwarkar et al., 2011). In the study of aerosol mycophenolate mofetil (MFF), rats received 50 mg/mL of MFF suspension for nebulization that was prepared from Cellcept®. The result presented that lung concentration of mycophenolic acid was estimated to 30% of serum concentration (Dugas et al., 2013). Besides, 150 mg of nintedanib (as mesylate) oral dose provided bioavailability 4.7%, plasma concentration 0.12 ng/mL/mg, and half-life at 9.49 hours (Marathe and Schuck, 2014). According to pharmacokinetic profile, the ratio of nintedanib, pirfenidone and mycophenolic acid is 1:3:3 respectively can be a possible dosage to provide therapeutic level via pulmonary delivery.

Thin-film freezing (TFF) is a particle engineering technology that can produce pharmaceutical powders by a rapid freezing process of drugs and excipients solution. TFF technique provides micro or nanoparticle powders of the formulation and decreases the ratio of air and water interface. However, this process has critical steps that have to be controlled including cryogen temperature, droplet velocity, the height of droplet to drum, and mass flow ratio of cryogen and liquid feed (Marathe and Schuck, 2014). Besides, the TFF technique can improve aerosol performance by modifying the surface texture. As a result, the preference powders for inhalation were produced by TFF process (Moon et al., 2019).

Nintedanib, pirfenidone and mycophenolic acid appear as useful drugs for pulmonary fibrosis especially idiopathic pulmonary fibrosis (IPF). However, oral drug delivery of two drugs of nintedanib, pirfenidone and mycophenolic acid exhibits low bioavailability, systemic adverse drug reactions and complicated dosage regimen. Importantly, three drug combination of nintedanib, pirfenidone and mycophenolic acid, may have more effective for IPF treatment, has not been established. In some embodiments, the present disclosure provides inhaled powders of fixed-dose drug combinations of nintedanib combined with pirfenidone or/and mycophenolic acid and single-drug inhaled dry powders of nintedanib, pirfenidone and mycophenolic acid prepared by thin-film freezing process.

These particles may be formulated in such a way that each of the particles contains two or more of the active pharmaceutical ingredients such as nintedanib, pirfenidone, and/or mycophenolic acid in the same particle. Some embodiments of the pharmaceutical compositions described herein the particles contain each of the active pharmaceutical ingredients. Furthermore, these pharmaceutical compositions may contain one or more properties that allow them to be delivered to the lungs through an inhaler. These particles show enhanced ability to break into smaller components. The particles may show a high surface area, a low tapped density, or a low bulk density. The surface area of the particles may be greater than 10 m²/g, greater than 25 m²/g, or greater than 50 m²/g. The bulk density of the particles may be less than 1 g/mL, less than 0.5 g/mL, or less than 0.25 g/mL. Finally, the tapped density of the particles may be less than 0.1 g/cm³, 0.05 g/cm³, or 0.025 g/cm³. Furthermore, these compositions may show improved flowability or compressibility such as a low Can's Index such as less than 20, less than 15, or less than 10.

In some embodiments, the particles comprise from about 10% w/w to about 80% w/w, from about 15% w/w to about 60% w/w, from about 20% w/w to about 40% w/w, from about 10% w/w to about 40% w/w, from about 20% w/w to about 30% w/w of the active pharmaceutical ingredients, from about 1% w/w to about 40% w/w of the active pharmaceutical ingredients, from about 5% w/w to about 20% w/w of the active pharmaceutical ingredients, from about 7.5% w/w to about 17.5% w/w of the active pharmaceutical ingredients, or about 5% w/w, 10% w/w, 15% w/w, 20% w/w, 21% w/w, 22% w/w, 23% w/w, 24% w/w, 25% w/w, 26% w/w, 27% w/w, 28% w/w, 29% w/w, 30% w/w, 31% w/w, 32% w/w, 33% w/w, 34% w/w, 35% w/w, 36% w/w, 37% w/w, 38% w/w, 39% w/w, 40% w/w, 45% w/w, or 50% w/w of the active pharmaceutical ingredients, or any range derivable therein.

In some embodiments, the particles comprise a weight ratio of nintedanib and pirfenidone from about 5:1 to about 1:10 or from about 1:1 to about 1:5, or from about 15:1, 14:1, 12:1, 10:1, 8:1, 6:1, 5:1, 4:1, 3:1, 2:1, 1:1:, 1:2, 1:4, 1:5, 1:6, 1:8, 1:10, 1:12, 1:14, or 1:15, or any range derivable therein. In some embodiments, the particles comprise a weight ratio of nintedanib and mycophenolic acid from about 5:1 to about 1:10 or from about 1:1 to about 1:5, or from about 15:1, 14:1, 12:1, 10:1, 8:1, 6:1, 5:1, 4:1, 3:1, 2:1, 1:1:, 1:2, 1:4, 1:5, 1:6, 1:8, 1:10, 1:12, 1:14, or 1:15, or any range derivable therein. In some embodiments, the particles comprise a weight ratio of pirfenidone and mycophenolic acid from about 10:1 to about 1:10 or from about 5:1 to about 1:5, or from about 15:1, 14:1, 12:1, 10:1, 8:1, 6:1, 5:1, 4:1, 3:1, 2:1, 1:1:, 1:2, 1:4, 1:5, 1:6, 1:8, 1:10, 1:12, 1:14, or 1:15, or any range derivable therein.

In some embodiments, the particles comprise at least 80% of one or more of the active pharmaceutical ingredients in the amorphous phase. In some embodiments, the amount of the one or more active pharmaceutical ingredients in the amorphous phase is from about 80%, 85%, 90%, 95%, 96%, 97%, 98%, 99%, 99.1%, 99.2%, 99.3%, 99.4%, 99.5%, 99.6%, 99.7%, 99.8%, to about 99.9%, or any range derivable therein.

In some embodiments, the particles containing nintedanib have a mass median aerodynamic diameter from about 1.0 μm to about 6.0 μm, from about 2.0 μm to about 5.0 μm, or from about 2.5 μm to about 4.5 μm. In some embodiments, the particles containing nintedanib have a mass median aerodynamic diameter from about 1.0 μm, 1.2 μm, 1.4 μm, 1.6 μm, 1.8 μm, 2.0 μm, 2.2 μm, 2.4 μm, 2.6 μm, 2.8 μm, 3.0 μm, 3.2 μm, 3.4 μm, 3.6 μm, 3.8 μm, 4.0 μm, 4.2 μm, 4.4 μm, 4.6 μm, 4.8 μm, 5.0 μm, 5.2 μm, 5.4 μm, 5.6 μm, 5.8 μm to about 6.0 μm, or any range derivable therein. In some embodiments, the particles containing pirfenidone have a mass median aerodynamic diameter from about 1.0 μm to about 7.0 μm, from about 2.0 μm to about 6.0 μm, or from about 3.0 μm to about 5.0 μm. In some embodiments, the particles containing pirfenidone have a mass median aerodynamic diameter from about 1.0 μm, 1.2 μm, 1.4 μm, 1.6 μm, 1.8 μm, 2.0 μm, 2.2 μm, 2.4 μm, 2.6 μm, 2.8 μm, 3.0 μm, 3.2 μm, 3.4 μm, 3.6 μm, 3.8 μm, 4.0 μm, 4.2 μm, 4.4 μm, 4.6 μm, 4.8 μm, 5.0 μm, 5.2 μm, 5.4 μm, 5.6 μm, 5.8 μm, 6.0 μm, 6.2 μm, 6.4 μm, 6.6 μm, 6.8 μm to about 7.0 μm, or any range derivable therein. In some embodiments, the particles containing mycophenolic acid have a mass median aerodynamic diameter from about 1.0 μm to about 6.0 μm, from about 1.5 μm to about 5.0 μm, or from about 2.0 μm to about 4.5 μm. In some embodiments, the particles containing mycophenolic acid have a mass median aerodynamic diameter from about 1.0 μm, 1.2 μm, 1.4 μm, 1.6 μm, 1.8 μm, 2.0 μm, 2.2 μm, 2.4 μm, 2.6 μm, 2.8 μm, 3.0 μm, 3.2 μm, 3.4 μm, 3.6 μm, 3.8 μm, 4.0 μm, 4.2 μm, 4.4 μm, 4.6 μm, 4.8 μm, 5.0 μm, 5.2 μm, 5.4 μm, 5.6 μm, 5.8 μm, to about 6.0 μm, or any range derivable therein. Copley Inhaler Testing Data Analysis Software (CITDAS) version 3.10 (Copley Scientific, Nottingham, UK) was used to calculate the aerodynamic particle size distribution including mass median aerodynamic diameter (MMAD), fine particle fraction (FPF), geometric standard deviation (GSD) and emitted fraction (EF). Mass median aerodynamic (MMAD) and geometric standard deviation (GSD) were evaluated by the cumulative percentage of mass and the aerodynamic diameter.

In some embodiments, the particles containing nintedanib have a geometric standard deviation (GSD) from about 1 to about 7.5, from about 1.5 to about 5.0, or form about 2 to about 4. In some embodiments, the particles containing nintedanib have a geometric standard deviation (GSD) from about 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8 to about 8.0, or any range derivable therein. In some embodiments, the particles containing pirfenidone have a geometric standard deviation (GSD) from about 1 to about 8, from about 1.5 to about 6.5, or form about 2 to about 5.5. In some embodiments, the particles containing pirfenidone have a geometric standard deviation (GSD) from about 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8 to about 8.0, or any range derivable therein. In some embodiments, the particles containing mycophenolic acid have a geometric standard deviation (GSD) from about 1 to about 7.5, from about 1.5 to about 5.0, or form about 2 to about 4. In some embodiments, the particles containing mycophenolic acid have a geometric standard deviation (GSD) from about 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8 to about 8.0, or any range derivable therein.

In some embodiments, the pharmaceutical composition has a fine particle fraction of recovered dose of the particles containing nintedanib is greater than 15%, greater than 20%, or greater than 25%. In some embodiments, the pharmaceutical composition has a fine particle fraction of recovered dose of the particles containing nintedanib is greater than 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the pharmaceutical composition has a fine particle fraction of recovered dose of the particles containing pirfenidone is greater than 15%, greater than 20%, or greater than 25%. In some embodiments, the pharmaceutical composition has a fine particle fraction of recovered dose of the particles containing pirfenidone is greater than 10%, 11%, 12%, 13%, 14%, 15%, 16%, 17%, 18%, 19%, 20%, 21%, 22%, 23%, 24%, 25%, 26%, 27%, 28%, 29%, or 30%. In some embodiments, the pharmaceutical composition has a fine particle fraction of recovered dose of the particles containing mycophenolic acid is greater than 15%, greater than 18%, or greater than 20%. In some embodiments, the pharmaceutical composition has a fine particle fraction of recovered dose of the particles containing mycophenolic acid is greater than 10%, 11%, 12%, 13%, 14%, 15%, 16%, 17%, 18%, 19%, 20%, 21%, 22%, 23%, 24%, 25%, 26%, 27%, 28%, 29%, or 30%. The fine particle fraction of recovered dose was calculated from a fine-particle dose divided by a total mass (recovered dose) while a fine particle fraction of delivered dose was calculated from a fine-particle dose divided by a delivered dose. The fine particle dose and fraction was calculated at a 5 μm cutoff. Moreover, the percentage recovery was calculated by a percentage of a total mass (recovered dose) that was collected through NGI divided by a loading dose.

In some embodiments, the pharmaceutical composition has a fine particle fraction of delivered dose of the particles containing nintedanib is greater than 25%, greater than 30%, or greater than 35%. In some embodiments, the pharmaceutical composition has a fine particle fraction of delivered dose of the particles containing nintedanib is greater than 20%, 21%, 22%, 23%, 24%, 25%, 26%, 27%, 28%, 29%, 30%, 31%, 32%, 33%, 34%, 35%, 36%, 37%, 38%, 39%, or 40%. In some embodiments, the pharmaceutical composition has a fine particle fraction of delivered dose of the particles containing pirfenidone is greater than 20%, greater than 25%, or greater than 30%. In some embodiments, the pharmaceutical composition has a fine particle fraction of delivered dose of the particles containing pirfenidone is greater than 10%, 11%, 12%, 13%, 14%, 15%, 16%, 17%, 18%, 19%, 20%, 21%, 22%, 23%, 24%, 25%, 26%, 27%, 28%, 29%, 30%, 31%, 33%, 33%, 34%, 35%, 36%, 37%, 38%, 39%, or 40%. In some embodiments, the pharmaceutical composition has a fine particle fraction of delivered dose of the particles containing mycophenolic acid is greater than 20%, greater than 25%, or greater than 30%. In some embodiments, the pharmaceutical composition has a fine particle fraction of delivered dose of the particles containing mycophenolic acid is greater than 10%, 11%, 12%, 13%, 14%, 15%, 16%, 17%, 18%, 19%, 20%, 21%, 22%, 23%, 24%, 25%, 26%, 27%, 28%, 29%, 30%, 31%, 33%, 33%, 34%, 35%, 36%, 37%, 38%, 39%, or 40%.

In some embodiments, the pharmaceutical compositions have a percentage recovery as a function of the loaded dose of the particles containing nintedanib greater than 60%, greater than 65%, or greater than 70%. In some embodiments, the pharmaceutical compositions have a percentage recovery as a function of the loaded dose of the particles containing nintedanib greater than 55%, 56%, 57%, 58%, 59%, 60%, 61%, 62%, 63%, 64%, 65%, 66%, 67%, 68%, 69%, 70%, 71%, 72%, 73%, 74%, or 75%. In some embodiments, the pharmaceutical compositions have a percentage recovery as a function of the loaded dose of the particles containing pirfenidone greater than 60%, greater than 65%, or greater than 70%. In some embodiments, the pharmaceutical compositions have a percentage recovery as a function of the loaded dose of the particles containing pirfenidone greater than 55%, 56%, 57%, 58%, 59%, 60%, 61%, 62%, 63%, 64%, 65%, 66%, 67%, 68%, 69%, 70%, 71%, 72%, 73%, 74%, or 75%. In some embodiments, the pharmaceutical compositions have a percentage recovery as a function of the loaded dose of the particles containing mycophenolic acid greater than 70%, greater than 75%, or greater than 80%. In some embodiments, the pharmaceutical compositions have a percentage recovery as a function of the loaded dose of the particles containing mycophenolic acid greater than 65%, 66%, 67%, 68%, 69%, 70%, 71%, 72%, 73%, 74%, 75%, 76%, 77%, 78%, 79%, 80%, 81%, 82%, 83%, 84%, or 85%.

In some embodiments, the pharmaceutical compositions have an emitted fraction of the particles containing nintedanib as measured by an NGI greater than 60%, greater than 65%, or greater than 70%. In some embodiments, the pharmaceutical compositions have an emitted fraction of the particles containing nintedanib as measured by an NGI greater than 55%, 56%, 57%, 58%, 59%, 60%, 61%, 62%, 63%, 64%, 65%, 66%, 67%, 68%, 69%, 70%, 71%, 72%, 73%, 74%, or 75%. In some embodiments, the pharmaceutical compositions have an emitted fraction of the particles containing pirfenidone as measured by an NGI greater than 60%, greater than 65%, or greater than 70%. In some embodiments, the pharmaceutical compositions have an emitted fraction of the particles containing pirfenidone as measured by an NGI greater than 55%, 56%, 57%, 58%, 59%, 60%, 61%, 62%, 63%, 64%, 65%, 66%, 67%, 68%, 69%, 70%, 71%, 72%, 73%, 74%, or 75%, the pharmaceutical compositions have an emitted fraction of the particles containing mycophenolic acid as measured by an NGI greater than 70%, greater than 75%, or greater than 80%. In some embodiments, the pharmaceutical compositions have an emitted fraction of the particles containing mycophenolic acid as measured by an NGI greater than 65%, 66%, 67%, 68%, 69%, 70%, 71%, 72%, 73%, 74%, 75%, 76%, 77%, 78%, 79%, 80%, 81%, 82%, 83%, 84%, or 85%. The emitted fraction (EF) was calculated as the total amount of the emitted dose from the device as a percentage of the total amount that was collected through NGI.

In some embodiments, the pharmaceutical composition comprises a dose of pirfenidone is from about 0.25 mg to about 10 mg, from about 0.5 mg to about 7.5 mg, from about 0.75 mg to about 5 mg, or from about 0.25 mg, 0.50 mg, 0.75 mg, 1.00 mg, 1.25 mg, 1.50 mg, 1.75 mg, 2.00 mg, 2.25 mg, 2.50 mg, 2.75 mg, 3.00 mg, 3.25 mg, 3.50 mg, 3.75 mg, 4.00 mg, 4.25 mg, 4.50 mg, 4.75 mg, 5.00 mg, 5.25 mg, 5.50 mg, 5.75 mg, 6.00 mg, 6.25 mg, 6.50 mg, 6.75 mg, 7.00 mg, 7.25 mg, 7.50 mg, 7.75 mg, 8.00 mg, 8.25 mg, 8.50 mg, 8.75 mg, 9.00 mg, 9.25 mg, 9.50 mg, 9.75 mg, to about 10.0 mg, or any range derivable therein. In some embodiments, the pharmaceutical composition comprises a dose of mycophenolic acid is from about 0.25 μg/mL to about 10 μg/mL, from about 0.5 μg/mL to about 7.5 μg/mL, from about 0.75 μg/mL to about 5 μg/mL, or from about 0.25 μg/mL, 0.50 μg/mL, 0.75 μg/mL, 1.00 μg/mL, 1.25 μg/mL, 1.50 μg/mL, 1.75 μg/mL, 2.00 μg/mL, 2.25 μg/mL, 2.50 μg/mL, 2.75 μg/mL, 3.00 μg/mL, 3.25 μg/mL, 3.50 μg/mL, 3.75 μg/mL, 4.00 μg/mL, 4.25 μg/mL, 4.50 μg/mL, 4.75 μg/mL, 5.00 μg/mL, 5.25 μg/mL, 5.50 μg/mL, 5.75 μg/mL, 6.00 μg/mL, 6.25 μg/mL, 6.50 μg/mL, 6.75 μg/mL, 7.00 μg/mL, 7.25 μg/mL, 7.50 μg/mL, 7.75 μg/mL, 8.00 μg/mL, 8.25 μg/mL, 8.50 μg/mL, 8.75 μg/mL, 9.00 μg/mL, 9.25 μg/mL, 9.50 μg/mL, 9.75 μg/mL, to about 10.0 μg/mL, or any range derivable therein.

1. Inhalation

In some embodiments, the present disclosure relates to respirable particles must be in the aerodynamic size range, such as mean median aerodynamic diameter of around 2 to 10 microns or 4 to 8 microns in aerodynamic diameter. In some embodiments, the present disclosure provides methods for the administration of the inhalable pharmaceutical composition provided herein using a device. Administration may be, but is not limited, to inhalation of pharmaceutical using an inhaler. In some embodiments, an inhaler is a simple passive dry powder inhaler (DPI), such as a Plastiape RS01 monodose DPI. In a simple dry powder inhaler, dry powder is stored in a capsule or reservoir and is delivered to the lungs by inhalation without the use of propellants.

In some embodiments, an inhaler is a single use, disposable inhaler such as a single-dose DPI, such as a DoseOne™, Spinhaler, Rotohaler®, Aerolizer®, or Handihaler. These dry powder inhaler may be a passive DPI. In some embodiments, an inhaler is a multidose DPI, such as a Plastiape RS02, Turbuhaler®, Twisthaler™, Diskhaler®, Diskus®, or Ellipta™. In some embodiments, the inhaler is Twincer®, Orbital®, TwinCaps®, Powdair, Cipla Rotahaler, DP Haler, Revolizer, Multi-haler, Twister, Starhaler, or Flexhaler®. In some embodiments, an inhaler is a plurimonodose DPI for the concurrent delivery of single doses of multiple medications, such as a Plastiape RS04 plurimonodose DPI. Dry powder inhalers have medication stored in an internal reservoir, and medication is delivered by inhalation with or without the use of propellants. Dry powder inhalers may require an inspiratory flow rate greater than 30 L/min for effective delivery, such as between about 30-120 L/min.

In some embodiments, the inhalable pharmaceutical composition is delivered as a propellant formulation, such as HFA propellants.

In some embodiments, the inhaler may be a metered dose inhaler. Metered dose inhalers deliver a defined amount of medication to the lungs in a short burst of aerosolized medicine aided by the use of propellants. Metered dose inhalers comprise three major parts: a canister, a metering valve, and an actuator. The medication formulation, including propellants and any required excipients, are stored in the canister. The metering valve allows a defined quantity of the medication formulation to be dispensed. The actuator of the metered dose inhaler, or mouthpiece, contains the mating discharge nozzle and typically includes a dust cap to prevent contamination.

In some embodiments, an inhaler is a nebulizer or a soft-mist inhaler such as those described in PCT Publication No. WO 1991/14468 and WO 1997/12687, which are incorporated herein by reference. A nebulizer is used to deliver medication in the form of an aerosolized mist inhaled into the lungs. The medication formulation be aerosolized by compressed gas, or by ultrasonic waves. A jet nebulizer is connected to a compressor. The compressor emits compressed gas through a liquid medication formulation at a high velocity, causing the medication formulation to aerosolize. Aerosolized medication is then inhaled by the patient. An ultrasonic wave nebulizer generates a high frequency ultrasonic wave, causing the vibration of an internal element in contact with a liquid reservoir of the medication formulation, which causes the medication formulation to aerosolize. Aerosolized medication is then inhaled by the patient. In some embodiments, the single use, disposable nebulizer may be used herein. A nebulizer may utilize a flow rate of between about 3-12 L/min, such as about 6 L/min. In some embodiments, the nebulizer is a dry powder nebulizer.

In some embodiments, the composition may be administered on a routine schedule. As used herein, a routine schedule refers to a predetermined designated period of time. The routine schedule may encompass periods of time which are identical, or which differ in length, as long as the schedule is predetermined. For instance, the routine schedule may involve administration four times a day, three times a day, twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In some embodiments, the pharmaceutical composition is administered once per day. In preferred embodiments, the pharmaceutical composition is administered less than once per day, such as every other day, every third day, or once per week. In some embodiments, a complete dose of the pharmaceutical composition is between 0.05-30 mg, such as 0.1-10, 0.25-5, 0.3-5, or 0.5-5 mg.

In some embodiments, the amount of the pharmaceutical composition of the nebulizer or inhaler may be provided in a unit dosage form, such as in a capsule, blister or a cartridge, wherein the unit dose comprises at least 0.05 mg of the pharmaceutical composition, such as at least 0.075 mg or 0.100 mg of the pharmaceutical composition per dose. In particular aspects, the unit dosage form does not comprise the administration or addition of any excipient and is merely used to hold the powder for inhalation (i.e., the capsule, blister, or cartridge is not administered). In some embodiments, the entire amount of the powder load may be administered in a high emitted dose, such as at least 1 mg, preferably at least 10 mg, even more preferably 50 mg. In some embodiments, administration of the powder load results in a high fine particle dose into the deep lung such as greater than 1 mg. Preferably, the fine particle dose into the deep lung is at least 5 mg, even more preferably at least 10 mg. In some embodiments, the dose may further comprise using a dose from a reservoir or non-unit dose form and the relevant dose is metered out from the device such as a nasal spray or turbuhaler.

2. Uses of Compositions

Several clinical indications would benefit from administration of composite compositions with enhanced bioavailability. These indications include lung diseases. In particular, the compositions may be used to treat lung diseases associated with lung inflammation or fibrosis. Some non-limiting examples of lung diseases which may be treated with the pharmaceutical compositions described herein include interstitial lung disease and idiopathic pulmonary fibrosis.

In some embodiments, the pharmaceutical composition may be used to treat one or more diseases or disorders in combination with one or more additional active agents. In particular, the pharmaceutical composition may be used in conjunction with another anti-inflammatory agent or active agent which reduces one or more symptoms of the lung disease.

3. Excipients

In some aspects, the present disclosure comprises one or more excipients formulated into pharmaceutical compositions. An “excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Furthermore, these compound may be used as diluents in order to obtain a dosage that can be readily measured or administered to a patient. Non-limiting examples of excipients include polymers, stabilizing agents, surfactants, surface modifiers, solubility enhancers, buffers, encapsulating agents, antioxidants, preservatives, nonionic wetting or clarifying agents, viscosity increasing agents, and absorption-enhancing agents.

In some embodiments, the amount of the excipient in the pharmaceutical composition is from about 50% w/w to about 95% w/w, from about 65% w/w to about 85% w/w, from about 75% w/w to about 95% w/w, or from about 87.5% w/w to about 92.5% w/w. In some embodiments, the amount of the excipient in the pharmaceutical composition is from about 50% w/w, 55% w/w, 60% w/w, 65% w/w, 70% w/w, 75% w/w, 80% w/w, 85% w/w, 90% w/w, to about 95% w/w, or any range derivable therein. In some embodiments, the amount of the excipient in the pharmaceutical composition is about 75% w/w, about 85% w/w or about 90% w/w. In some embodiments, at least 80% of the excipient is in the amorphous phase. In some embodiments, the amount of the excipient in the amorphous phase is at least 80%, 85%, 90%, 95%, 96%, 97%, 98%, 99%, 99.1%, 99.2%, 99.3%, 99.4%, 99.5%, 99.6%, 99.7%, 99.8%, to about 99.9%, or any range derivable therein. In some embodiments, at least 80% of the excipient is in the crystalline phase. In some embodiments, the amount of the excipient in the crystalline phase is at least 80%, 85%, 90%, 95%, 96%, 97%, 98%, 99%, 99.1%, 99.2%, 99.3%, 99.4%, 99.5%, 99.6%, 99.7%, 99.8%, to about 99.9%, or any range derivable therein.

In some aspects, the pharmaceutical compositions of the present disclosure may further comprise one or more excipient, such as a sugar or sugar alcohol, an amino acid, lecithin, or a polymer. Additionally, cyclodextrin compounds such a sulfo ethyl β cyclodextrin may be used as excipients. Furthermore, one or more flow enhancing agents such as magnesium salts may be used. Some non-limiting examples of flow enhancing agents include magnesium stearate, sodium stearyl fumarate, a phospholipid such distearoylphosphatidyl-choline and L-leucine. Some composition may further comprise a mixture of two or more excipients.

1. Saccharides

In some aspects, the present disclosure comprises one or more excipients formulated into pharmaceutical compositions. In some embodiments, the excipients used herein are water soluble excipients. These saccharides may be used to act as a lyoprotectant that protects the protein from destabilization during the drying process. These water-soluble excipients include carbohydrates or saccharides such as disaccharides such as sucrose, trehalose, or lactose, a trisaccharide such as fructose, glucose, galactose comprising raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol. In other aspects, larger molecules like amino acids, peptides and proteins are incorporated to facilitate inhalation delivery, including leucin, trileucine, histidine and others. Some non-limiting examples of amino acids include hydrophobic amino acids, such as leucine. In some embodiments, the excipient may be lecithin.

2. Polymers

In some embodiments, the excipient is a pharmaceutically acceptable polymer. In some embodiments, the excipient is a non-cellulosic polymer. In some embodiments, the excipient is a non-ionizable non cellulosic polymer, such as polyvinylpyrrolidone. In some embodiments, the polyvinylpyrrolidone has a molecular weight from about 10,000 to about 40,000 or from about 20,000 to about 30,000. In some embodiments, the polyvinylpyrrolidone has a molecular weight from about 10,000, 12,000, 14,000, 16,000, 18,000, 20,000, 22,000, 24,000, 26,000, 28,000, 30,000, 32,000, 34,000, 36,000, 38,000, to about 40,000, or any range derivable therein. In some embodiments the polyvinylpyrrolidone has a molecular weight of about 24,000.

### II. MANUFACTURING METHODS

A. Thin-Film Freezing

The final formulations may also be prepared using a thin-film freezing technique. Without wishing to be bound by any theory, it is believed that this process may be used to introduce the particles into a single particle containing one or more active pharmaceutical ingredients. In particular, if multiple therapeutic agents are present in the composition, the particles contain two or more of the active pharmaceutical ingredients. The particles obtained from this process may exhibit one or more beneficial properties for administration via inhalation such as a high surface area, a low tapped density, a low bulk density, or improved flowability or compressibility such as a low Can's Index. In some aspects, the present disclosure provides methods of preparing a pharmaceutical composition of the present disclosure comprising:
(A) dissolving an active pharmaceutical ingredient in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a pharmaceutical composition.

In some embodiments, the pharmaceutical mixture comprises a solid content of the active pharmaceutical ingredient and the excipient from about 0.05% w/v to about 5% w/v, from about 0.1% w/v to about 2.5% w/v, 0.15% w/v to about 1.5% w/v, 0.2% w/v to about 0.6% w/v, 0.5% w/v to about 1.25% w/v, or from about 0.050% w/v, 0.075% w/v, 0.10% w/v, 0.125% w/v, 0.150% w/v, 0.175% w/v, 0.200% w/v, 0.225% w/v, 0.250% w/v, 0.275% w/v, 0.300% w/v, 0.325% w/v, 0.350% w/v, 0.375% w/v, 0.400% w/v, 0.425% w/v, 0.450% w/v, 0.475% w/v, 0.500% w/v, 0.525% w/v, 0.550% w/v, 0.575% w/v, to about 0.600% w/v, or any range derivable therein. In some embodiments, the pharmaceutical mixture is applied at a feed rate from about 0.50 mL/min to about 5.00 mL/min, from about 1.00 mL/min to about 3.00 mL/min, or from about 0.500 mL/min, 0.750 mL/min, 1.00 mL/min, 1.25 mL/min, 1.50 mL/min, 1.75 mL/min, 2.00 mL/min, 2.25 mL/min, 2.50 mL/min, 2.75 mL/min, 3.00 mL/min, 3.25 mL/min, 3.50 mL/min, 3.75 mL/min, 4.00 mL/min, 4.25 mL/min, 4.50 mL/min, 4.75 mL/min, to about 5.00 mL/min, or any range derivable therein.

In some embodiments, the pharmaceutical mixture is applied from a height from about 2 cm to about 50 cm, from about 5 cm to about 20 cm, or from about 1 cm, 2 cm, 4 cm, 6 cm, 8 cm, 10 cm, 12 cm, 14 cm, 16 cm, 18 cm, 20 cm, 21 cm, 22 cm, 23 cm, 24 cm, to about 25 cm, or any range derivable therein. In some embodiments, the surface temperature is from about −190° C. to about 0° C., from about −125° C. to about −25° C., or from about −190° C., −180° C., −170° C., −160° C., −150° C., −140° C., −130° C., −120° C., −110° C., −100° C., −90° C., −80° C., −70° C., −60° C., −50° C., −40° C., −30° C., −20° C., −10° C., to about 0° C., or any range derivable therein. In some embodiments, the surface is rotating at a speed from about 5 rpm to about 500 rpm, from about 100 rpm to about 400 rpm, or from about 5 rpm, 10 rpm, 15 rpm, 25 rpm, 50 rpm, 75 rpm, 100 rpm, 150 rpm, 200 rpm, 250 rpm, 300 rpm, 350 rpm, 400 rpm, 450 rpm, to about 500 rpm, or any range derivable therein.

In some embodiments, the wherein the frozen pharmaceutical composition is dried by lyophilization. In further embodiments, the frozen pharmaceutical composition is dried at a first reduced pressure from about 10 mTorr to 500 mTorr, from about 50 mTorr to about 250 mTorr, or from about 10 mTorr, 20 mTorr, 30 mTorr, 40 mTorr, 50 mTorr, 75 mTorr, 100 mTorr, 150 mTorr, 200 mTorr, 250 mTorr, 300 mTorr, 350 mTorr, 400 mTorr, 450 mTorr, to about 500 mTorr, or any range derivable therein. In some embodiments, the frozen pharmaceutical composition is dried at a first reduced temperature from about −100° C. to about 0° C., from about −60° C. to about −20° C., or from about −100° C., −90° C., −80° C., −70° C., −60° C., −50° C., −40° C., −30° C., −20° C., −10° C., to about 0° C., or any range derivable therein. In some embodiments, the frozen pharmaceutical composition is dried for a primary drying time period from about 3 hours to about 36 hours, from about 6 hours to about 24 hours, or from about 3 hours, 4 hours, 5 hours, 6 hours, 12 hours, 18 hours, 24 hours, 30 hours, to about 36 hours, or any range derivable therein.

In some embodiments, the frozen pharmaceutical composition is dried at a second reduced pressure from about 10 mTorr to 500 mTorr, from about 50 mTorr to about 250 mTorr, or from about 10 mTorr, 20 mTorr, 30 mTorr, 40 mTorr, 50 mTorr, 75 mTorr, 100 mTorr, 150 mTorr, 200 mTorr, 250 mTorr, 300 mTorr, 350 mTorr, 400 mTorr, 450 mTorr, to about 500 mTorr, or any range derivable therein. In some embodiments, the frozen pharmaceutical composition is dried at a second reduced temperature from about 0° C. to about 30° C., from about 10° C. to about 30° C., or from about 0° C., 5° C., 10° C., 15° C., 20° C., 25° C., to about 30° C., or any range derivable therein. In some embodiments, the frozen pharmaceutical composition is dried for a second time for a second time period from about 3 hours to about 36 hours, from about 6 hours to about 24 hours, or from about 3 hours, 4 hours, 5 hours, 6 hours, 12 hours, 18 hours, 24 hours, 30 hours, to about 36 hours, or any range derivable therein. In some embodiments, the temperature is changed from the first reduced temperature to the second reduced temperature over a ramping time period from about 3 hours to about 36 hours, from about 6 hours to about 24 hours, or from about 3 hours, 4 hours, 5 hours, 6 hours, 12 hours, 18 hours, 24 hours, 30 hours, to about 36 hours, or any range derivable therein.

### III. DEFINITIONS

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified polysaccharide, wherein at least one of the monomeric saccharide units is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example hydrochlorides, hydrobromides, sulfates, hydrogen sulfates or phosphates, salts with suitable carboxylic acids, such as optionally hydroxylated lower alkanoic acids, for example acetic acid, glycolic acid, propionic acid, lactic acid or pivalic acid, optionally hydroxylated and/or oxo-substituted lower alkanedicarboxylic acids, for example oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example methanesulfonates, benzenesulfonates, p-toluenesulfonates or N-cyclohexylsulfamates (cyclamates).

The term “dissolution” as used herein refers to a process by which a solid substance, here the active ingredients, is dispersed and then dissolved in molecular form in a medium. The dissolution rate of the active ingredients of the pharmaceutical dose of the invention is defined by the amount of drug substance that goes in solution per unit time under standardized conditions of liquid/solid interface, temperature and solvent composition.

As used herein, the term “aerosols” refers to dispersions in air of solid or liquid particles, of fine enough particle size and consequent low settling velocities to have relative airborne stability (See Knight, V., Viral and Mycoplasmal Infections of the Respiratory Tract. 1973, Lea and Febiger, Phila. Pa., pp. 2).

As used herein, the term “physiological pH” refers to a solution with is at its normal pH in the average human. In most situation, the solution has a pH of approximately 7.4.

As used herein, “inhalation” or “pulmonary inhalation” is used to refer to administration of pharmaceutical preparations by inhalation so that they reach the lungs and in particular embodiments the alveolar regions of the lung. Typically inhalation is through the mouth, but in alternative embodiments in can entail inhalation through the nose.

As used herein, “dry powder” refers to a fine particulate composition that is not suspended or dissolved in an aqueous liquid.

A “simple dry powder inhaler” refers a device for the delivery of medication to the respiratory tract, in which the medication is delivered as a dry powder in a single-use, single-dose manner. In particular aspects, a simple dry powder inhaler has fewer than 10 working parts. In some aspects, the simple dry powder inhaler is a passive inhaler such that the dispersion energy is provided by the patient's inhalation force rather than through the application of an external energy source.

A “median particle diameter” refers to the geometric diameter as measured by laser diffraction or image analysis. In some aspects, at least either 50% or 80% of the particles by volume are in the median particle diameter range.

A “Mass Median Aerodynamic Diameter (MMAD)” refers to the aerodynamic diameter (different than the geometric diameter) and is measured by laser diffraction.

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active agent in the composition is measured by powder x-ray diffraction.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±5% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “essentially free of” or “essentially free” is used to represent that the composition contains less than 1% of the specific component. The term “entirely free of” or “entirely free” contains less than 0.1% of the specific component.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Inhaled Drug Combinations of Nintedanib, Pirfenidone, and Mycophenolic Acid

Materials. Drug combinations for the treatment of pulmonary fibrosis include nintedanib, pirfenidone and mycophenolic acid. Nintedanib esylate was purchased from Ontario Chemicals Inc. Pirfenidone was purchased from Oakwood Products, Inc, and Mycophenolic acid was purchased from AK Scientific.

Dry Powder Inhaler Preparations. Dry powders for inhalation were prepared by thin-film freezing (TFF) technique. Nintedanib esylate, pirfenidone, and mycophenolic acid were dissolved in acetonitrile and water mixture and then the mixtures were sonicated for 5 minutes to obtain a clear solution. Afterward, stabilizers and other excipients were added in the solution and sonicated for 5 minutes to obtain the clear solution. Total volume of the solution is 30 mL, and solid content was 0.5% w/v. The compositions of each formulation were showed in Table 1. Approximately 15 microlites of each formulation was dropped at 10 cm height onto a rotating drum that was stainless steel cooled by cryogenic. Liquid nitrogen was used to control temperature through TFF process at −70° C. to −90° C. The frozen disks of samples were collected into a stainless-steel chamber filled with liquid nitrogen and preserved in a −80° C. freezer before transferring to a lyophilizer. A lyophilizer was used to dry frozen samples by removing the solvents. The samples were primary dried at −40° C. for 20 hours, then ramped to 25° C. over 20 hours and secondary dried at 25° C. for 20 hours for drying process. Pressure during the drying process was controlled at 100 mTorr.

TABLE 1
Compositions of the formulations.

| | | | | | | Total |
|---|---|---|---|---|---|---|
| | 0.5% w/v solid content | | | | | vol- |
| Formu- | | % | Ex- | % | | ume |
| lations | Drug | w/w | cipient | w/w | Diluent | (mL) |
| T01 | Nintedanib | 3.57 | Lactose | 75.00 | Water:ACN | 30 |
| | Pirfenidone | 10.71 | | | (1:1) | |
| | Myco- | 10.71 | | | | |
| | phenolic | | | | | |
| | acid | | | | | |
| T02 | Nintedanib | 3.57 | Man- | 75.00 | Water:ACN | 30 |
| | Pirfenidone | 10.71 | nitol | | (1:1) | |
| | Myco- | 10.71 | | | | |
| | phenolic | | | | | |
| | acid | | | | | |
| T03 | Nintedanib | 3.57 | Man- | 70.00 | Water:ACN | 30 |
| | Pirfenidone | 10.71 | nitol | | (1:1) | |
| | Myco- | 10.71 | Lec- | 5.00 | | |
| | phenolic | | ithin | | | |
| | acid | | | | | |
| T04 | Nintedanib | 3.57 | Man- | 73.00 | Water:ACN | 30 |
| | Pirfenidone | 10.71 | nitol | | (1:1) | |
| | Myco- | 10.71 | PVP | 2.00 | | |
| | phenolic | | K25 | | | |
| | acid | | | | | |
| F06 | Nintedanib | 6.25 | Lactose | 75.00 | Water:ACN | 30 |
| | Pirfenidone | 18.75 | | | (1:1) | |
| F07 | Nintedanib | 6.25 | Man- | 75.00 | Water:ACN | 30 |
| | Pirfenidone | 18.75 | nitol | | (1:1) | |
| F08 | Nintedanib | 6.25 | Man- | 70.00 | Water:ACN | 30 |
| | Pirfenidone | 18.75 | nitol | | (1:1) | |
| | | | Lec- | 5.00 | | |
| | | | ithin | | | |
| F09 | Nintedanib | 6.25 | Man- | 73.00 | Water:ACN | 30 |
| | Pirfenidone | 18.75 | nitol | 2.00 | (1:1) | |
| | | | PVP | | | |
| | | | K25 | | | |
| NM01 | Nintedanib | 6.25 | Lactose | 75.00 | Water:ACN | 30 |
| | Myco- | 18.75 | | | (1:1) | |
| | phenolic | | | | | |
| | acid | | | | | |
| NM02 | Nintedanib | 6.25 | Man- | 75.00 | Water:ACN | 30 |
| | Myco- | 18.75 | nitol | | (1:1) | |
| | phenolic | | | | | |
| | acid | | | | | |
| NM03 | Nintedanib | 6.25 | Man- | 70.00 | Water:ACN | 30 |
| | Myco- | 18.75 | nitol | | (1:1) | |
| | phenolic | | Lec- | 5.00 | | |
| | acid | | ithin | | | |
| NM04 | Nintedanib | 6.25 | Man- | 73.00 | Water:ACN | 30 |
| | Myco- | 18.75 | nitol | | (1:1) | |
| | phenolic | | PVP | 2.00 | | |
| | acid | | K25 | | | |

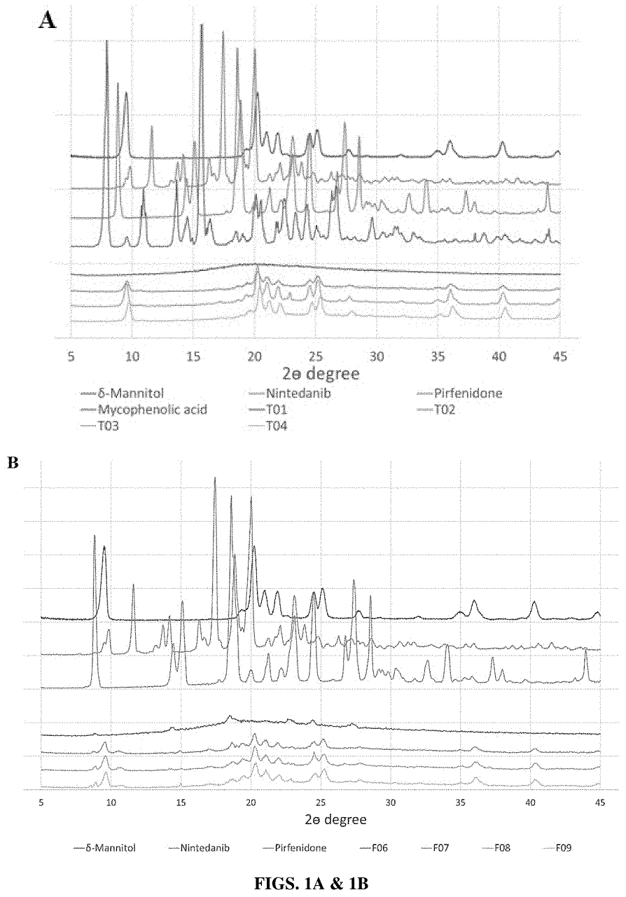

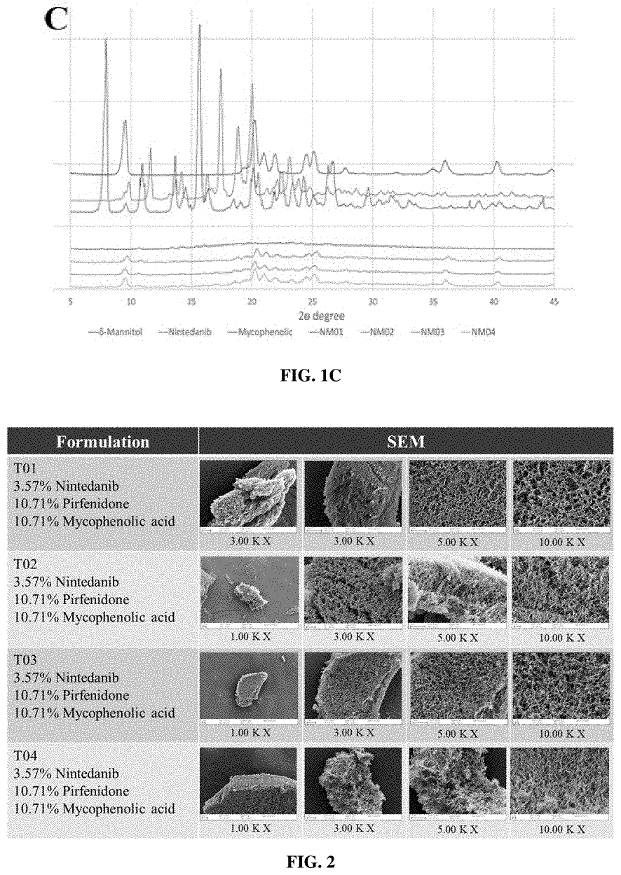

Physicochemical Properties. The amorphous structure of TFF powders was investigated by X-ray Powder Diffraction (XRPD). X-ray diffraction (MiniFlex 600, Rigaku Co., Tokyo, Japan) measure from 5 to 45° over a 20 range (0.02° step, 3°/min, 40 kV, 15 mA). No peak was observed in T01 which indicates drugs and lactose were amorphous while F06 showed small peaks of pirfenidone and NM01 showed small peaks of nintedanib (FIGS. 1A-1C). In other formulations including T02, T03, T04, F07, F08, F09, NM02, NM03, and NM04, XRD diffractograms showed that mannitol was crystalline, while drugs and other excipients were amorphous (FIGS. 1A-1C).

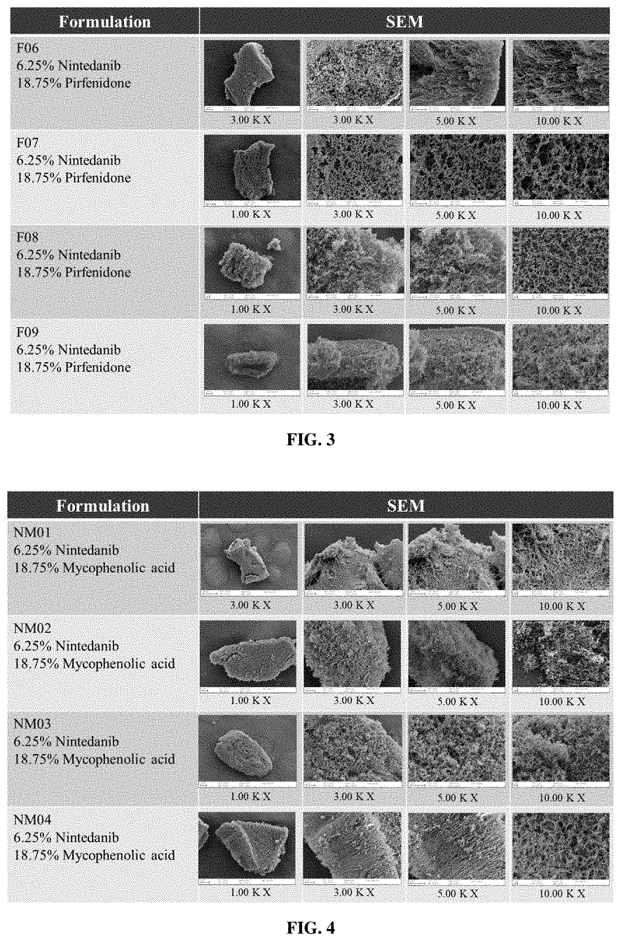

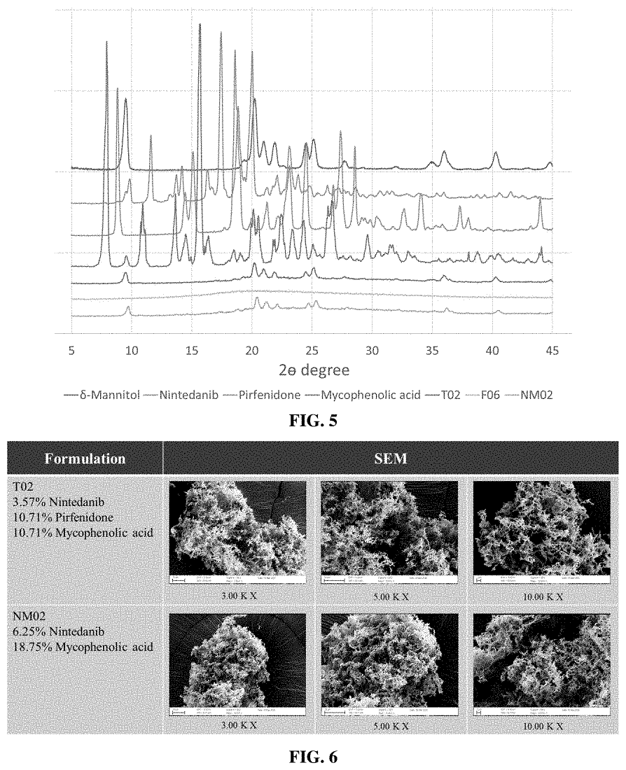

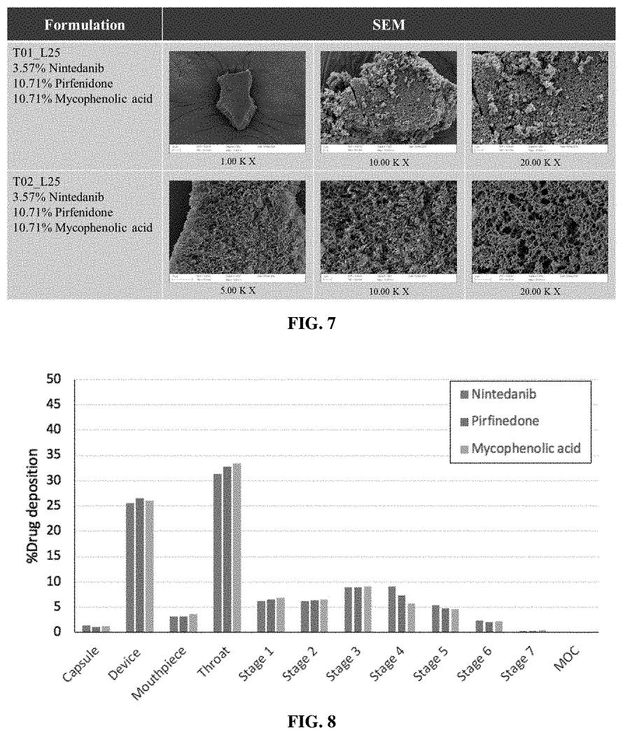

The surface morphology of TFF powder was identified by Scanning Electron Microscopy (SEM) (Zeiss Supra 40 V SEM, Carl Zeiss, Heidenheim an der Brenz, Germany). TFF powder was placed onto carbon tape and sputter coated with 15 mm thickness of platinum/palladium alloy (Pt/Pd) before capturing the images. In three drug combinations, T01, T02, T03, T04 showed homogenous TFF-formulation. T01 (75% Lactose) and T02 (75% Mannitol) showed the matrix structure (FIG. 2). In two drugs combination, F06, F07, F08, F09 showed matrix structure and homogenous formulations (FIG. 3). Moreover, NM01, NM02, NM03, NM04 showed brittle matrix structure and homogenous formulations (FIG. 4).

### Example 2—Confirmation of Results and Improvement in Recovery of NGI Performing

Materials. Drugs for pulmonary fibrosis including nintedanib esylate, pirfenidone and mycophenolic acid. Nintedanib esylate was purchased from Ontario Chemicals Inc. Pirfenidone was purchased from Oakwood Products, Inc, and Mycophenolic acid was purchased from AK Scientific. Moreover, lactose and mannitol were used as stabilizers.

Dry powder inhaler preparations. Dry powders for inhalation were prepared by thin-film freezing (TFF) technique. Nintedanib esylate, pirfenidone, and mycophenolic acid were dissolved in acetonitrile and water mixture and then the mixtures were sonicated for 5 minutes to obtain a clear solution. Afterward, lactose or mannitol were added in the solution and sonicated for 5 minutes to obtain the clear solution. Total volume of the solution is 40 mellites, and solid content was 0.5% w/v. The compositions of each formulation were showed in Table 2. Approximately 15 microlites of each formulation was dropped at 10 cm height onto a rotating drum that was stainless steel cooled by cryogenic. Liquid nitrogen was used to control temperature through TFF process at −70° C. to −90° C. The frozen disks of samples were collected into a stainless-steel chamber filled with liquid nitrogen and preserved in a −80° C. freezer before transferring to a lyophilizer. A lyophilizer was used to dry frozen samples by removing the solvents. The samples were primary dried at −40° C. for 20 hours, then ramped to 25° C. over 20 hours and secondary dried at 25° C. for 20 hours for drying process. Pressure during the drying process was controlled at 100 mTorr.

TABLE 2
Compositions of each formulation.

| | | | | | | Total |
|---|---|---|---|---|---|---|
| | 0.5% w/v solid content | | | | | vol- |
| Formu- | | % | Ex- | % | | ume |
| lations | Drug | w/w | cipient | w/w | Diluent | (mL) |
| T01 | Nintedanib | 3.57 | Lactose | 75.00 | Water:ACN | 40 |
| | Pirfenidone | 10.71 | | | (1:1) | |
| | Myco- | 10.71 | | | | |
| | phenolic | | | | | |
| | acid | | | | | |
| T02 | Nintedanib | 3.57 | Man- | 75.00 | Water:ACN | 40 |
| | Pirfenidone | 10.71 | nitol | | (1:1) | |
| | Myco- | 10.71 | | | | |
| | phenolic | | | | | |
| | acid | | | | | |
| F06 | Nintedanib | 6.25 | Lactose | 75.00 | Water:ACN | 40 |
| | Pirfenidone | 18.75 | | | (1:1) | |
| NM01 | Nintedanib | 6.25 | Lactose | 75.00 | Water:ACN | 40 |
| | Myco- | 18.75 | | | (1:1) | |
| | phenolic | | | | | |
| | acid | | | | | |
| NM02 | Nintedanib | 6.25 | Man- | 75.00 | Water:ACN | 40 |
| | Myco- | 18.75 | nitol | | (1:1) | |
| | phenolic | | | | | |
| | acid | | | | | |

Physicochemical Properties. The amorphous structure of TFF powders was investigated by X-ray Powder Diffraction (XRPD). X-ray diffraction (MiniFlex 600, Rigaku Co., Tokyo, Japan) measure from 5 to 45° over a 20 range (0.02° step, 3°/min, 40 kV, 15 mA). T01 and NM01 showed amorphous structure of nintedanib, pirfenidone and mycophenolic acid. Other formulations including T02 and NM02 showed amorphous structure of nintedanib, pirfenidone and mycophenolic acid with mannitol peak (FIG. 5).

The surface morphology of TFF powders was investigated by Scanning Electron Microscopy (SEM) (Zeiss Supra 40 V SEM, Carl Zeiss, Heidenheim an der Brenz, Germany). TFF powders were placed onto carbon tape and sputter coated with 15 mm thickness of platinum/palladium alloy (Pt/Pd) for 20 minutes before capturing the images. In three-drug combination, T02 showed brittle matrix and homogenous TFF powders. In two-drug combination, NM02 showed matrix and homogenous powders (FIG. 6).

Aerodynamic particle size distribution. Aerodynamic particle size distribution was investigated by a Next Generation Pharmaceutical Impactor (NGI) (MSP Co. Shoreview, Minn.) connected with High Capacity Pump (model HCP5, Copley Scientific, Nottingham, UK), and Critical Flow Controller (model TPK 2000, Copley Scientific, Nottingham, UK) was used to control air flow rate. A HPMC capsule size 3 containing TFF powders (approximately 7 to 8 mg per capsules), was loaded into high resistant RS00 dry powder inhaler (Plastiape, Osnago, Italy). TFF powders (15 mg) were delivered into the NGI through the USP induction port, stages 1-7, and micro-orifice collector (MOC) at the flow rate of 60 L/min for 4 s per each actuation. The deposited powders from the capsule, inhaler, adapter, induction port, stages 1-7, and the micro-orifice collector (MOC) were collected by diluting with a mixture of acetonitrile and water (50/50 v/v).

The drug content was analyzed with a Thermo Scientific™ Dionex™ UltiMate™ 3000 HPLC system (Thermo Scientific, Sunnyvale, Calif., USA) with a Waters Xbridge C18 column (4.6×150 mm, 3.5 μm) (Milford, Mass.). A gradient method that was 25% of acetonitrile to 7 minutes, 60% of acetonitrile to 8 minutes, and 25% of Acetonitrile to 9 minutes was also used to detect the content of nintedanib, pirfenidone, and mycophenolic acid. The retention time of three drugs were 5.80 minutes for nintedanib, 4.51 minutes for pirfenidone, and 7.36 minutes for mycophenolic acid.

In three-drug combination, T01 showed MMAD of nintedanib at 3.12 μm, pirfenidone at 3.39 μm and mycophenolic acid at 3.57 μm. Besides, T02 showed MMAD of nintedanib at 4.16 μm, pirfenidone at 4.83 μm and mycophenolic acid at 4.07 μm. In the two-drug combinations, F06 showed MMAD of nintedanib at 2.89 μm and MMAD of pirfenidone at 4.08 μm. NM01 showed MMAD of nintedanib at 2.53 μm and MMAD of mycophenolic acid at 2.49 μm. NM02 showed MMAD of nintedanib at 4.10 μm and MMAD of mycophenolic acid at 4.23 μm (Table 3).

TABLE 3
Mass Median Aerodynamic Diameter (MMAD) result of
the combination products.

| | MMAD (μm) | | |
|---|---|---|---|
| | | | Mycophenolic |
| Formulation | Nintedanib | Pirfenidone | acid |
| T01 | 3.12 | 3.39 | 3.57 |
| T02 | 4.16 | 4.83 | 4.07 |
| F06 | 2.89 | 4.08 | |
| NM01 | 2.53 | | 2.49 |
| NM02 | 4.10 | | 4.23 |

In FPF results of recovered dose, T01 containing lactose showed the FPF at 28.89%, 28.08%, and 20.74% of nintedanib, pirfenidone, and mycophenolic acid, respectively. T02 containing mannitol showed FPF at 49.39%, 28.21%, 49.52% of nintedanib, pirfenidone, and mycophenolic acid correspondingly. F06 containing lactose showed FPF of nintedanib at 48.85% and FPF of pirfenidone was 30.19%. In addition, NM01 showed the FPF at 57.08% and 58.32%, of nintedanib and mycophenolic acid correspondingly. NM02 showed the FPF at 48.56% and 45.52% of nintedanib and mycophenolic acid respectively. (Table 4).

TABLE 4
Fine particle fraction (FPF) of recovered dose.

| | FPF (of recovered dose) | | |
|---|---|---|---|
| | | | Mycophenolic |
| Formulation | Nintedanib | Pirfenidone | acid |
| T01 | 28.89 | 28.08 | 20.74 |
| T02 | 49.39 | 28.21 | 49.52 |
| F06 | 48.85 | 30.19 | |
| NM01 | 57.08 | | 58.32 |
| NM02 | 48.56 | | 45.52 |

In the FPF results of delivered dose, T01 containing lactose showed the FPF at 47.72%, 46.93%, and 37.89% of nintedanib, pirfenidone, and mycophenolic acid correspondingly. T02 containing mannitol showed FPF at 55.40%, 31.26%, 55.50% of nintedanib, pirfenidone, and mycophenolic acid correspondingly. F06 containing lactose showed FPF of nintedanib at 57.50% and FPF of pirfenidone was 36.98%. In addition, NM01 showed FPF at 65.69% and 66.18%, of nintedanib and mycophenolic acid correspondingly. NM02 showed FPF of nintedanib at 51.06% and FPF of mycophenolic acid at 50.10% (Table 5).

TABLE 5
Fine particle fraction (FPF) of delivered dose.

| | FPF (of delivered dose) | | |
|---|---|---|---|
| | | | Mycophenolic |
| Formulation | Nintedanib | Pirfenidone | acid |
| T01 | 47.72 | 46.93 | 37.89 |
| T02 | 55.40 | 31.26 | 55.50 |
| F06 | 57.50 | 36.98 | |
| NM01 | 65.69 | | 66.18 |
| NM02 | 51.06 | | 50.10 |

TABLE 6
Recovery of NGI.

| | % Recovery (of loaded dose) | | |
|---|---|---|---|
| | | | Mycophenolic |
| Formulation | Nintedanib | Pirfenidone | acid |
| T01 | 70.60 | 70.40 | 88.15 |
| T02 | 73.99 | 82.92 | 88.52 |

### Example 3—Recovery of NGI Performing and Increase % FPF

Materials. Drugs for pulmonary fibrosis including nintedanib esylate, pirfenidone and mycophenolic acid. Nintedanib esylate was purchased from Ontario Chemicals Inc. Pirfenidone was purchased from Oakwood Products, Inc, and Mycophenolic acid was purchased from AK Scientific. Moreover, lactose and mannitol were used as stabilizers.

Dry Powder Inhaler Preparations. Dry powders for inhalation were prepared by thin-film freezing (TFF) technique. Nintedanib esylate, pirfenidone, and mycophenolic acid were prepared as stock solutions in acetonitrile and water mixture and then aliquot to prepare each formulation. Afterward, lactose or mannitol were added in the formulations and sonicated for 5 minutes to obtain the clear solution. Solid content was 0.5% w/v. The compositions of each formulation were showed in Table 7. Approximately 15 μL of each formulation was dropped at 10 cm height onto a rotating drum that was stainless steel cooled by cryogenic. Liquid nitrogen was used to control temperature through TFF process at −70° C. to −90° C. The frozen disks of samples were collected into a stainless-steel chamber filled with liquid nitrogen and preserved in a −80° C. freezer before transferring to a lyophilizer. A lyophilizer was used to dry frozen samples by removing the solvents. The samples were primary dried at −40° C. for 20 hours, then ramped to 25° C. over 20 hours and secondary dried at 25° C. for 20 hours for drying process. Pressure during the drying process was controlled at 100 mTorr.

TABLE 7
Compositions of each formulation.

| | 0.5% w/v solid content | | | | |
|---|---|---|---|---|---|
| Formulation | Drug | % w/w | Excipient | % w/w | Diluent |
| T01 | Nintedanib | 3.57 | Lactose | 75.00 | Water:ACN |
| | Pirfenidone | 10.71 | | | (1:1) |
| | Mycophenolic | 10.71 | | | |
| | acid | | | | |
| T02 | Nintedanib | 3.57 | Mannitol | 75.00 | Water:ACN |
| | Pirfenidone | 10.71 | | | (1:1) |
| | Mycophenolic | 10.71 | | | |
| | acid | | | | |
| T01_L25 | Nintedanib | 3.57 | Lactose | 50.00 | Water: ACN |
| | Pirfenidone | 10.71 | Leucine | 25.00 | (1:1) |
| | | 10.71 | | | |
| T02_L25 | Nintedanib | 3.57 | Mannitol | 50.00 | Water:ACN |
| | Mycophenolic | 10.71 | Leucine | 25.00 | (1:1) |
| | acid | | | | |

Physicochemical Properties. The surface morphology of TFF powders was investigated by Scanning Electron Microscopy (SEM) (Zeiss Supra 40 V SEM, Carl Zeiss, Heidenheim an der Brenz, Germany). TFF powders were placed onto carbon tape and sputter coated with 15 mm thickness of platinum/palladium alloy (Pt/Pd) before capturing the images. In three-drug combination, T01_L25 and T02_L25 showed brittle matrix and homogenous TFF powders (FIG. 7).

Aerodynamic particle size distribution. Aerodynamic particle size distribution was investigated by a Next Generation Pharmaceutical Impactor (NGI) (MSP Co. Shoreview, Minn.) connected with High Capacity Pump (model HCP5, Copley Scientific, Nottingham, UK), and Critical Flow Controller (model TPK 2000, Copley Scientific, Nottingham, UK) was used to control air flow rate. A HPMC capsule size 3 containing TFF powders was loaded into high resistant RS00 dry powder inhaler (Plastiape, Osnago, Italy). TFF powders (15 mg) were delivered into the NGI through the USP induction port, stages 1-7, and micro-orifice collector (MOC) at the flow rate of 60 L/min for 4 s per each actuation. The deposited powders from the capsule, inhaler, adapter, induction port, stages 1-7, and the micro-orifice collector (MOC) were collected by diluting with a mixture of acetonitrile and water (50/50 v/v).

The drug content was analyzed with a Thermo Scientific™ Dionex™ UltiMate™ 3000 HPLC system (Thermo Scientific, Sunnyvale, Calif., USA) with a Waters Xbridge C18 column (4.6×150 mm, 3.5 μm) (Milford, Mass.). A gradient method that was 25% of acetonitrile to 7 minutes, 60% of acetonitrile to 8 minutes, and 25% of Acetonitrile to 9 minutes was also used to detect the content of nintedanib, pirfenidone, and mycophenolic acid. The retention time of three drugs were 5.80 minutes for nintedanib, 4.51 minutes for pirfenidone, and 7.36 minutes for mycophenolic acid.

Aerodynamic performance of T01 showed MMAD of nintedanib at 3.12 μm, pirfenidone at 3.39 μm, and mycophenolic acid at 3.57 μm. FPF (of recovery dose) of nintedanib, pirfenidone and mycophenolic acid were 25.88%, 25.00% and 23.75%, respectively (Table 8). In T02, MMAD of nintedanib was 4.16 μm and pirfenidone was 4.83 μm, while mycophenolic acid was 4.07 μm. FPF (of recovery dose) of nintedanib, pirfenidone and mycophenolic acid were 45.16%, 37.94% and 48.75%, respectively (Table 9).

TABLE 8
Aerosol performance of T01.

| | | | Mycophenolic |
|---|---|---|---|
| T01 | Nintedanib | Pirfenidone | acid |
| MMAD | 3.12 | 3.39 | 3.57 |
| GSD | 2.15 | 2.10 | 2.02 |
| FPF (of recovered dose) | 25.88 | 25.00 | 23.75 |
| FPF (of delivered dose) | 35.41 | 34.52 | 32.64 |
| EF (of recovered dose) | 73.08 | 72.42 | 72.75 |
| % Recovery (of loaded | 93.84 | 75.65 | 93.52 |
| dose) | | | |

TABLE 9
Aerosol performance of T02.

| | | | Mycophenolic |
|---|---|---|---|
| T02 | Nintedanib | Pirfenidone | acid |
| MMAD | 4.16 | 4.83 | 4.07 |
| GSD | 3.64 | 4.16 | 3.51 |
| FPF (of recovered dose) | 45.16 | 37.94 | 48.75 |
| FPF (of delivered dose) | 48.06 | 41.82 | 55.50 |
| EF (of recovered dose) | 93.97 | 90.72 | 91.49 |
| % Recovery (of loaded | 92.33 | 61.02 | 95.45 |
| dose) | | | |

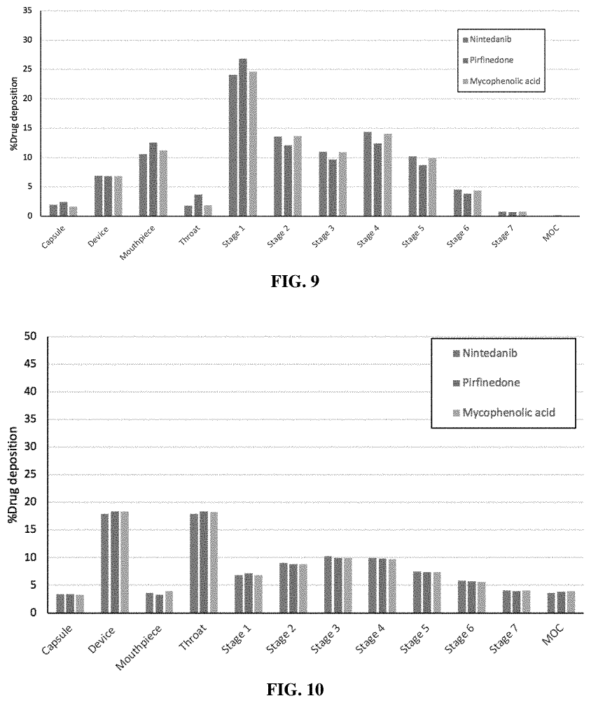

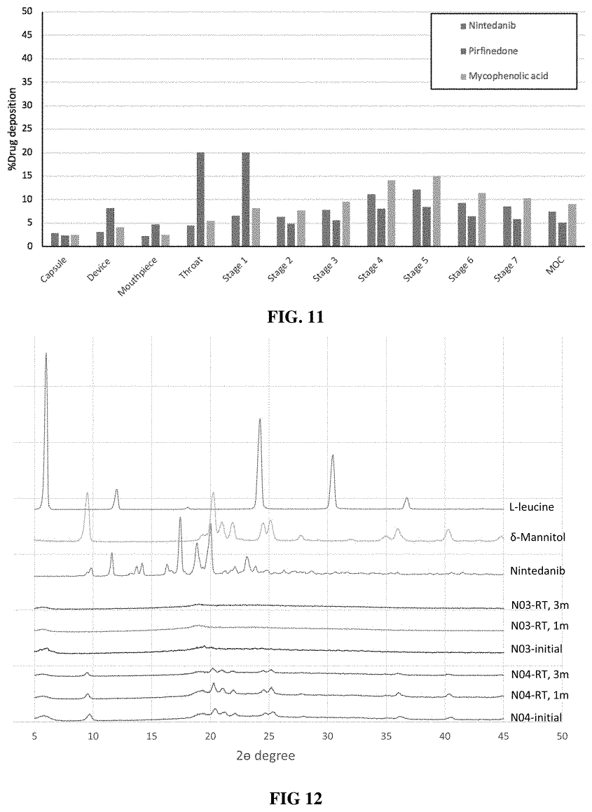

Aerodynamic performance of T01_L25 showed MMAD of nintedanib at 2.48 μm, pirfenidone at 2.49 μm, and mycophenolic acid at 2.45 μm. FPF (of recovery dose) of nintedanib, pirfenidone and mycophenolic acid were 43.31%, 43.37% and 42.70%, respectively (Table 10). In T02_L25, MMAD of nintedanib was 1.51 μm and pirfenidone was 2.54 μm, while mycophenolic acid was 1.50 μm. FPF (of recovery dose) of nintedanib, pirfenidone and mycophenolic acid were 70.61%, 40.54% and 69.35%, respectively (Table 11). In T01, T02 and T01_L25, drug deposition of each drug showed similar distribution between drugs in every stage of NGI (FIGS. 8-10), while in T02_L25, drug deposition of pirfenidone showed higher distribution between drugs in the throat and stage 1 (FIG. 11).

TABLE 10
Aerosol performance of T01_L25.

| | | | Mycophenolic |
|---|---|---|---|
| T01_L25 | Nintedanib | Pirfenidone | acid |
| MMAD | 2.48 | 2.49 | 2.45 |
| GSD | 3.32 | 3.33 | 3.33 |
| FPF (of recovered dose) | 43.31 | 43.37 | 42.70 |
| FPF (of delivered dose) | 55.06 | 54.54 | 54.49 |
| EF (of recovered dose) | 78.67 | 79.52 | 78.35 |
| % Recovery (of loaded | 95.52 | 74.69 | 99.74 |
| dose) | | | |

TABLE 11
Aerosol performance of T02_L25.

| | | | Mycophenolic |
|---|---|---|---|
| T02_L25 | Nintedanib | Pirfenidone | acid |
| MMAD | 1.51 | 2.54 | 1.50 |
| GSD | 3.99 | 5.34 | 3.59 |
| FPF (of recovered dose) | 70.61 | 40.54 | 69.35 |
| FPF (of delivered dose) | 76.17 | 45.40 | 76.27 |
| EF (of recovered dose) | 92.70 | 89.30 | 90.94 |
| % Recovery (of loaded | 87.78 | 70.60 | 87.10 |
| dose) | | | |

### Example 4—Inhaled Nintedanib Compositions, Characteristics and Aerodynamic Properties

Materials. Nintedanib containing nintedanib esylate was purchased from Ontario Chemicals Inc.

Dry Powder Inhaler Preparations. Dry powders for inhalation were prepared by thin-film freezing (TFF) technique. Nintedanib esylate was dissolved in 50% v/v acetonitrile and water mixture and sonicated for 5 minutes to obtain a clear solution. Stabilizers and other excipients were separately dissolved in water and sonicated for 5 minutes to obtain the clear solution. Afterward, aliquot the drug solution and excipient solutions into a bottle and then add water and acetonitrile to obtain total volume in 50% v/v acetonitrile and water mixture. The compositions of each formulation were showed in Table 12. Approximately 15 microlites of each formulation was dropped at 10 cm height onto a rotating drum that was stainless steel cooled by cryogenic. Liquid nitrogen was used to control temperature through TFF process at −70° C. to −90° C. The frozen disks of samples were collected into a stainless-steel chamber filled with liquid nitrogen and preserved in a −80° C. freezer before transferring to a lyophilizer. A lyophilizer was used to dry frozen samples by removing the solvents. The samples were primary dried at −40° C. for 20 hours, then ramped to 25° C. over 20 hours and secondary dried at 25° C. for 20 hours for drying process. Pressure during the drying process was controlled at 100 mTorr.

TABLE 12
Compositions of each formulation.

| Formula- | 0.5% w/v solid content | | | | |
|---|---|---|---|---|---|
| tions | Drug | % w/w | Excipient | % w/w | Diluent |
| N03 | Nintedanib | 20.00 | Lactose | 55.00 | Water:ACN |
| | | | Leucine | 25.00 | (1:1) |
| N04 | Nintedanib | 50.00 | Mannitol | 55.00 | Water:ACN |
| | | | Leucine | 25.00 | (1:1) |
| N14 | Nintedanib | 50.00 | Captisol ® | 50.00 | Water:ACN |
| | | | | | (1:1) |
| N15 | Nintedanib | 50.00 | Lactose | 50.00 | Water:ACN |
| | | | | | (1:1) |
| N17 | Nintedanib | 50.00 | Lactose | 25.00 | Water:ACN |
| | | | Leucine | 25.00 | (1:1) |
| N18 | Nintedanib | 50.00 | Leucine | 50.00 | Water:ACN |
| | | | | | (1:1) |

Stability study. The formulations containing 20% w/w of nintedanib (N03 and N04) were storage at room temperature (i.e., 25° C.) for 1 and 3 months. The formulations containing 50% w/w of nintedanib (N14, N15, N17 and N18) were stored at 40° C. for 2 weeks. Each formulation at each time point was investigated for their physicochemical and aerodynamic properties.

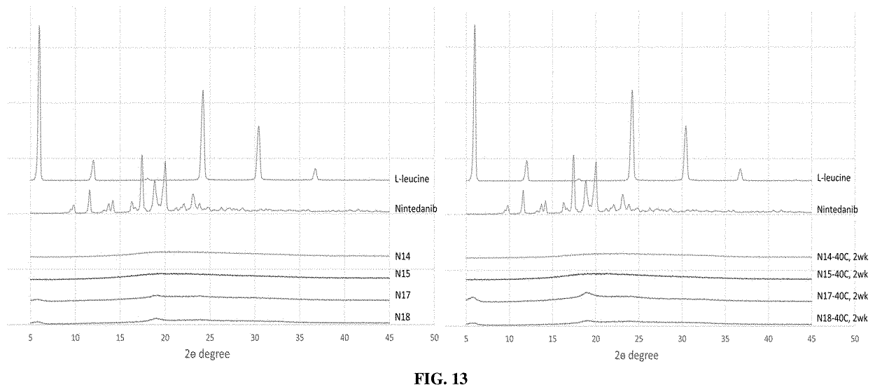

Physicochemical Properties. The amorphous morphology of TFF powders was investigated by X-ray Powder Diffraction (XRPD). X-ray diffraction (MiniFlex 600, Rigaku Co., Tokyo, Japan) measure from 5° to 45° over a 2θ range (0.02° step, 3°/min, 40 kV, 15 mA). Inhaled nintedanib formulations including N03, N14, N15, N17 and N18 showed amorphous morphology of nintedanib and excipients (FIG. 13). However, N04 showed amorphous morphology of nintedanib with mannitol peak (FIG. 12). After storage at room temperature for 1 and 3 months, inhaled nintedanib formulations including N03 and N04 also showed amorphous morphology of nintedanib. In term of 50% nintedanib formulations storage at 40° C. for 2 weeks, N14, N15, N17 and N18 showed no different change of amorphous morphology of nintedanib and excipients (FIG. 13).

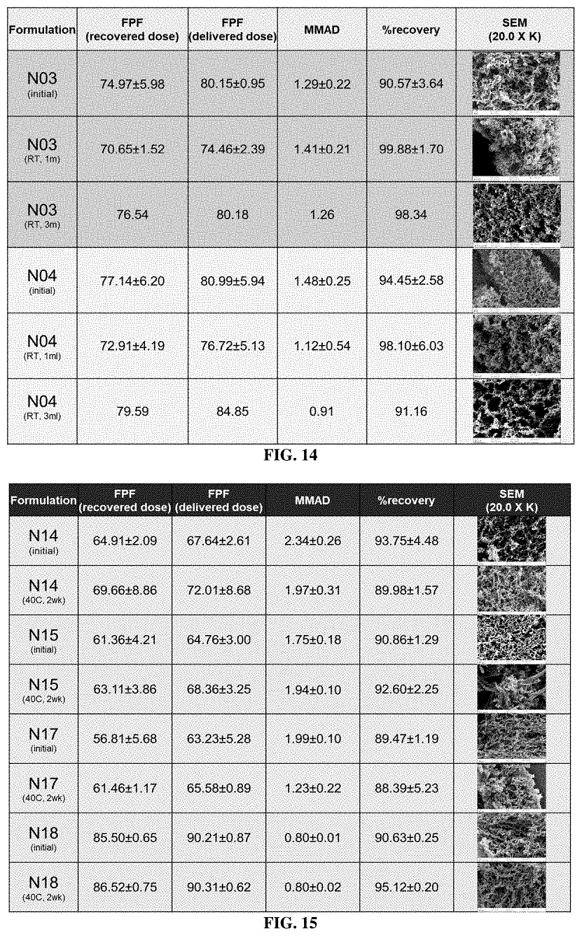

The surface morphology of TFF powders was investigated by Scanning Electron Microscopy (SEM) (Zeiss Supra 40 V SEM, Carl Zeiss, Heidenheim an der Brenz, Germany). TFF powders were placed onto carbon tape and sputter coated with 15 mm thickness of platinum/palladium alloy (Pt/Pd) before capturing the images. In inhaled nintedanib formulations, N03, N04, N14, N15, N17 and N18 showed brittle matrix and homogenous TFF powders at initial point (FIG. 14-15). After storage condition, inhaled nintedanib formulations including N03, N04, N14, N17 and N18 showed no different in brittle matrix and homogenous TFF powders compared to initial point. However, N15 containing 50% w/w lactose showed higher particles and slightly lower homogenous structure of the powders compared to initial point. (FIG. 14-15).

Aerodynamic particle size distribution. Aerodynamic particle size distribution was investigated by a Next Generation Pharmaceutical Impactor (NGI) (MSP Co. Shoreview, Minn.) connected with High Capacity Pump (model HCP5, Copley Scientific, Nottingham, UK), and Critical Flow Controller (model TPK 2000, Copley Scientific, Nottingham, UK) was used to control air flow rate. A HPMC capsule size 3 containing TFF powders (approximately 5 mg per capsules), was loaded into high resistant RS00 dry powder inhaler (Plastiape, Osnago, Italy). TFF powders (5 mg) were delivered into the NGI through the USP induction port, stages 1-7, and micro-orifice collector (MOC) at the flow rate of 60 L/min for 4 s per each actuation. The deposited powders from the capsule, inhaler, adapter, induction port, stages 1-7, and the micro-orifice collector (MOC) were collected by diluting with a mixture of acetonitrile and water (50/50 v/v).

The drug content was analyzed with a Thermo Scientific™ Dionex™ UltiMate™ 3000 HPLC system (Thermo Scientific, Sunnyvale, Calif., USA) with a Waters Xbridge C18 column (4.6×150 mm, 3.5 μm) (Milford, Mass.). A gradient method that was 25% of acetonitrile to 7 minutes, 60% of acetonitrile to 8 minutes, and 25% of Acetonitrile to 9 minutes was also used to detect the content of nintedanib. The retention time of nintedanib was 5.80 minutes.

In the results of aerodynamic properties, N03 and N04 containing 20% w/w of nintedanib showed MMAD at 1.29 μm and 1.12 μm respectively (FIG. 14). In term of 50% w/w nintedanib formulations, N18 containing 50% w/w leucine showed the lowest MMAD at 0.80 μm, while N14 containing 50% w/w lactose showed the highest MMAD at 2.34 μm. N15 containing lactose showed MMAD at 1.75 μm and N17 containing lactose and leucine showed MMAD of nintedanib at 1.99 μm (FIG. 15). After storage at room temperature for 1 and 3 months, N03 containing 20% w/w of nintedanib showed MMAD at 1.41 μm and 1.26 μm respectively compared similar to an initial point at 1.29 μm. N04 showed MMAD at 1.12 μm (1 month) and 0.91 μm (3 months) compared to initial point at 1.48 μm (FIG. 14). In term of 50% w/w nintedanib formulations, inhaled nintedanib formulations also showed high aerodynamic properties after storage at 40° C. for 2 weeks. N18 containing 50% w/w leucine showed the lowest MMAD at 0.80 μm, while N14 containing 50% w/w Captisol® showed the highest MMAD at 1.97 μm compared to initial point at 2.34 μm. N15 containing lactose showed MMAD of nintedanib at 1.94 μm and N17 containing lactose and leucine showed MMAD at 1.23 μm (FIG. 15).

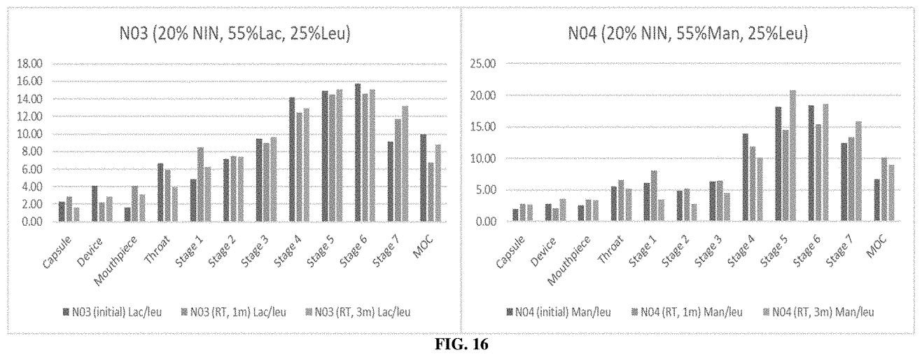

In FPF results of recovered dose, N03 containing lactose showed lower FPF at 74.97% compared with N04 containing mannitol (FPF at 77.14%). In FPF results of delivered dose, N03 also showed lower FPF at 80.15% compared with N04 that showed the FPF at 80.99% (FIG. 14). After storage at room temperature for 1 and 3 months, N03 containing 20% w/w of nintedanib showed high FPF of recovered dose at 70.65% and 76.54% respectively compared to an initial point at 74.97%. N04 showed FPF of recovered dose at 72.91% (1 month) and 79.50% (3 months) similar to an initial point at 77.14% In FPF results of delivered dose, N03 also showed FPF at 74.46% (1 month) and 80.18% (3 month) similar to the initial point at 80.15%. N04 showed FPF of delivered dose at 76.72% (1 month) and 84.85% (3 months) similar to an initial point at 80.99% (FIG. 14). In addition, N03 showed similarly drug deposition compared with the initial point, while N04 at 3 month showed higher deposition at stage 5 to MOC compared to an initial point and 1 month (FIG. 16).

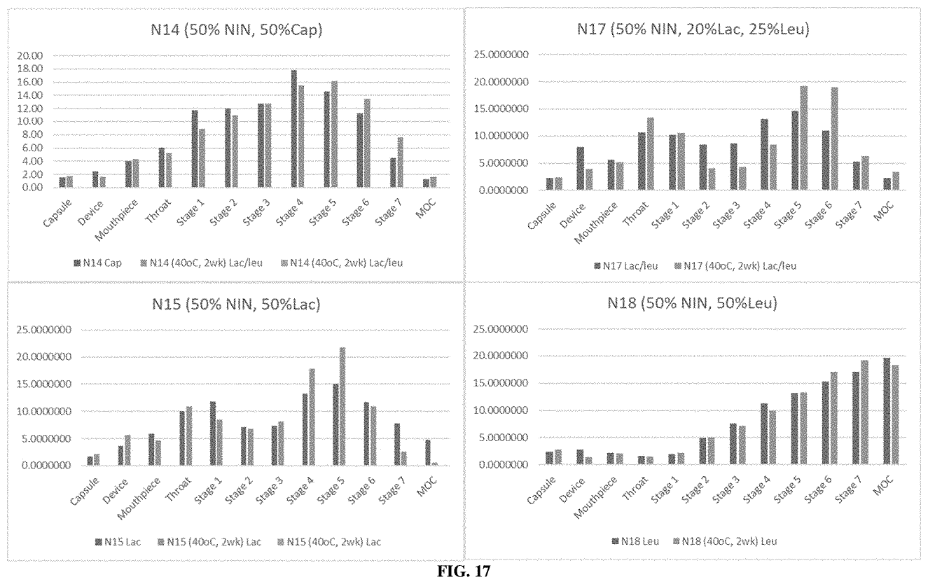

In terms of 50% w/w nintedanib, N18 containing leucine showed the highest FPF of recovered dose at 85.50%, while N17 containing lactose and leucine showed the lowest FPF at 56.81%. Moreover, N14 containing Captisol® and N15 containing lactose showed FPF of recovered dose at 64.91% and 61.36% correspondingly (FIG. 15). After storage at 40° C. for 2 weeks, the inhaled nintedanib formulations showed high FPF of recovered dose as the initial point. N18 containing leucine showed the highest FPF of recovered dose at 86.52% similar to an initial point (85.50%), while N17 containing lactose and Captisol® showed the lowest FPF at 61.46% higher than an initial point at 56.81%. Furthermore, N14 containing Captisol® and N15 containing lactose showed high FPF of recovered dose at 69.66% and 63.11% correspondingly (FIG. 15). In addition, N14 and N18 showed similar drug deposition to the initial point. N15 and N17 containing lactose showed different drug deposition compared to the initial point (FIG. 17).

### Example 5—Study of Aerodynamic Performance

Materials. Nintedanib esylate, pirfenidone and mycophenolic acid were used. Nintedanib esylate was purchased from Ontario Chemicals Inc. Pirfenidone was purchased from Oakwood Products, Inc, and Mycophenolic acid was purchased from AK Scientific. Moreover, lactose, mannitol, and leucine were used as stabilizers.

Dry Powder Inhaler Preparations. Dry powders for inhalation were prepared by thin-film freezing (TFF) technique. In T10, T11, NM08 and NM09 formulations, nintedanib esylate (NIN), pirfenidone (PIR), and mycophenolic acid (MA) were prepared as stock solutions in acetonitrile and water mixture and then aliquoted to prepare each formulation. Afterward, lactose, mannitol or leucine were added in the formulations and sonicated for 5 minutes to obtain the clear solution. Solid content was 0.5% w/v. In T35-T40 formulations, pirfenidone and mycophenolic acid were prepared as stock solutions in acetonitrile and then aliquoted to prepare PIR-MA solution that was sonicated for 15 minutes. Nintedanib esylate were prepared as stock solutions in acetonitrile and water mixture and then aliquot to PIR-MA solution to obtain a drug solution. In T40 formulation, fumaric acid (FA) was dissolved in water and aliquot to prepare PIR-FA solutions that was sonicated for 15 minutes before adding NIN and MA. Afterward, lactose was added in the drug solution and sonicated for 15 minutes to obtain the clear solution. Solid content was 0.1% w/v. The compositions of each formulation were showed in Table 13. Approximately 15 μL of each formulation was dropped at 10 cm height onto a rotating drum that was stainless steel cooled by cryogenic. Liquid nitrogen was used to control temperature through TFF process at −100° C. to −120° C. The frozen disks of samples were collected into a stainless-steel chamber filled with liquid nitrogen and preserved in a −80° C. freezer before transferring to a lyophilizer. A lyophilizer was used to dry frozen samples by removing the solvents. The samples were primary dried at −40° C. for 20 hours, then ramped to 25° C. over 20 hours and secondary dried at 25° C. for 20 hours for drying process. Pressure during the drying process was controlled at 100 mTorr.

TABLE 13
Compositions of each formulation.

| | | | | | Solid | |
|---|---|---|---|---|---|---|
| | | | | | content | |
| Formulation | Drug | % w/w | Excipient | % w/w | (% w/v) | Diluent |
| T10 | Nintedanib | 3.57 | Lactose | 25.00 | 0.5 | Water:ACN |
| | Pirfenidone | 10.71 | Leucine | 50.00 | | (1:1) |
| | Mycophenolic acid | 10.71 | | | | |
| T11 | Nintedanib | 3.57 | Leucine | 75.00 | 0.5 | Water:ACN |
| | Pirfenidone | 10.71 | | | | (1:1) |
| | Mycophenolic acid | 10.71 | | | | |
| T35 | Nintedanib | 3.57 | Mannitol | 75.00 | 0.1 | Water:ACN |
| | Pirfenidone | 10.71 | | | | (1:1) |
| | Mycophenolic acid | 10.71 | | | | |
| T36 | Nintedanib | 3.57 | Lactose | 75.00 | 0.1 | Water:ACN |
| | Pirfenidone | 10.71 | | | | (1:1) |
| | Mycophenolic acid | 10.71 | | | | |
| T37 | Nintedanib | 3.57 | Lactose | 25.00 | 0.1 | Water:ACN |
| | Pirfenidone | 10.71 | Leucine | 50.00 | | (1:1) |
| | Mycophenolic acid | 10.71 | | | | |
| T38 | Nintedanib | 4.76 | Lactose | 66.67 | 0.1 | Water:ACN |
| | Pirfenidone | 14.29 | | | | (1:1) |
| | Mycophenolic acid | 14.29 | | | | |
| T40 | Nintedanib | 3.57 | Lactose | 68.39 | 0.1 | Water:ACN |
| | Pirfenidone | 10.71 | Fumaric acid | 6.71 | | (1:1) |
| | Mycophenolic acid | 10.71 | | | | |
| NM08 | Nintedanib | 6.25 | Lactose | 50.00 | 0.5 | Water:ACN |
| | Mycophenolic acid | 18.75 | Leucine | 25.00 | | (1:1) |
| NM09 | Nintedanib | 6.25 | Mannitol | 50.00 | 0.5 | Water:ACN |
| | Mycophenolic acid | 18.75 | Leucine | 25.00 | | (1:1) |

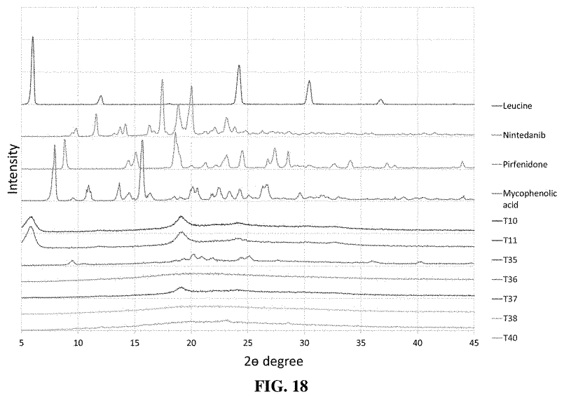

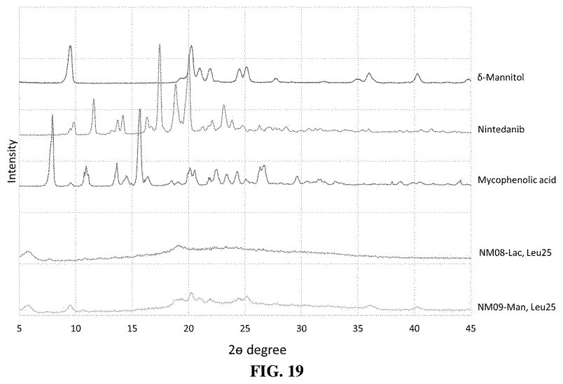

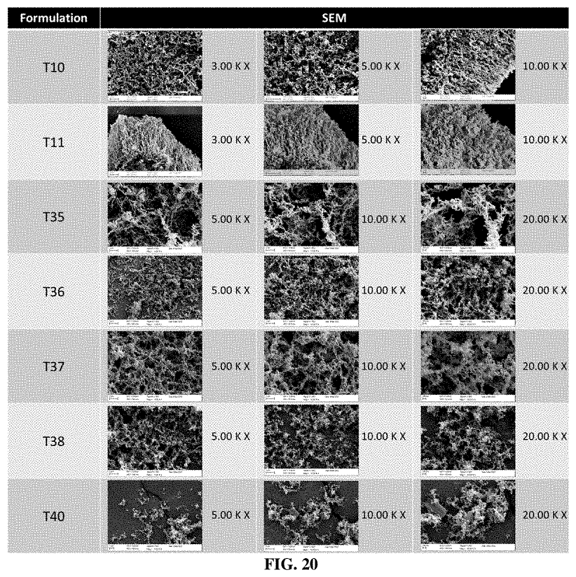

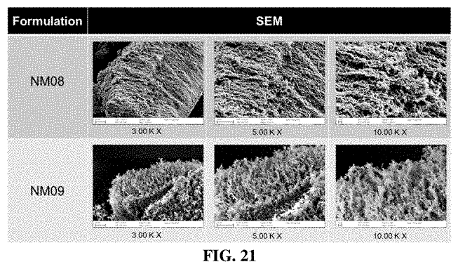

Physicochemical Properties. The amorphous morphology of TFF powders was investigated by X-ray Powder Diffraction (XRPD). X-ray diffraction (MiniFlex 600, Rigaku Co., Tokyo, Japan) measure from 5° to 45° over a 2θ range (0.04° step, 2°/min, 40 kV, 15 mA). In triple-drug combination formulations, T36, T38 and T40 showed amorphous morphology of nintedanib, pirfenidone, mycophenolic acid and excipients. However, T10, T11 and T37 showed peaks of pirfenidone and leucine that presented crystalline morphology (FIG. 18). In NIN-MA formulations, NM08 and NM09 showed amorphous morphology of nintedanib and mycophenolic acid, while crystalline peaks of leucine and mannitol were shown in FIG. 19.

The surface morphology of TFF powders was investigated by Scanning Electron Microscopy (SEM) (Zeiss Supra 40 V SEM, Carl Zeiss, Heidenheim an der Brenz, Germany). TFF powders were placed onto carbon tape and sputter coated with 15 mm thickness of platinum/palladium alloy (Pt/Pd) before capturing the images. In triple-drug combination, T10-T11 and T35-T38 showed brittle matrix and homogenous TFF powders, while T40 showed pirfenidone particle about 1-2 μm dispersed in brittle matrix powders (FIG. 20). In NIN-MA formulations, NM08 and NM09 showed brittle matrix and homogenous TFF powders. Primary particles size was shown below 1 μm (FIG. 21).

Aerodynamic particle size distribution. Aerodynamic particle size distribution was investigated by a Next Generation Pharmaceutical Impactor (NGI) (MSP Co. Shoreview, Minn.) connected with High Capacity Pump (model HCP5, Copley Scientific, Nottingham, UK), and Critical Flow Controller (model TPK 2000, Copley Scientific, Nottingham, UK) was used to control air flow rate. A HPMC capsule size 3 containing TFF powders was loaded into high resistant RS00 dry powder inhaler (Plastiape, Osnago, Italy). TFF powders (15 mg) were delivered into the NGI through the USP induction port, stages 1-7, and micro-orifice collector (MOC) at the flow rate of 60 L/min for 4 s per each actuation. The deposited powders from the capsule, inhaler (device), adapter (mouthpiece), induction port (throat), stages 1-7, and the micro-orifice collector (MOC) were collected by diluting with a mixture of acetonitrile and water (50/50% v/v).

The drug content was analyzed with a Thermo Scientific™ Dionex™ UltiMate™ 3000 HPLC system (Thermo Scientific, Sunnyvale, Calif., USA) with a Waters Xbridge C18 column (4.6×150 mm, 3.5 μm) (Milford, Mass.). A gradient method that was 25% of acetonitrile to 6 minutes, 60% of acetonitrile to 8 minutes, and 25% of Acetonitrile to 9 minutes was also used to detect the content of nintedanib, pirfenidone, and mycophenolic acid. The retention time of three drugs were 5.80 minutes for nintedanib, 4.53 minutes for pirfenidone, and 7.36 minutes for mycophenolic acid.

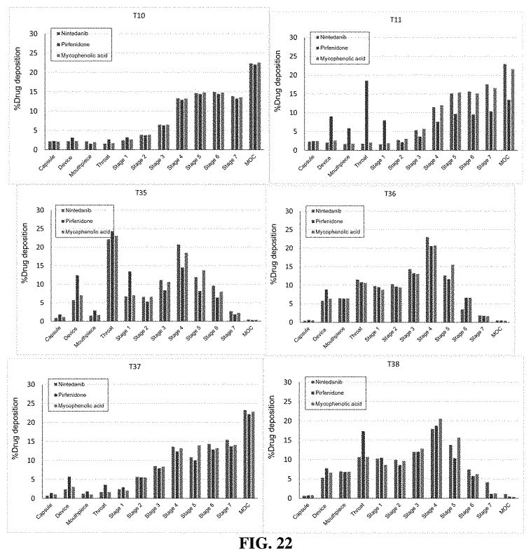

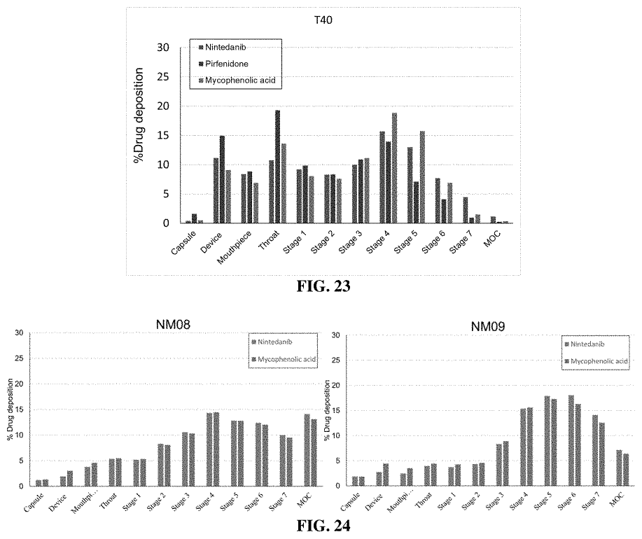

Aerodynamic performance of T10 containing lactose and leucine showed the lowest MMAD of nintedanib at 0.80 μm, pirfenidone at 0.81 μm, and mycophenolic acid at 0.80 μm. The highest FPF (of recovery dose) of nintedanib, pirfenidone and mycophenolic acid were 86.54%, 84.28% and 86.43%, respectively. T37 containing lactose and leucine showed the lowMMAD of nintedanib at 0.82 μm, pirfenidone at 0.83 μm, and mycophenolic acid at 0.84 μm. The high FPF (of recovery dose) of nintedanib, pirfenidone and mycophenolic acid were 87.32%, 80.35% and 87.12%, respectively. T35 and T36 showed MMAD of nintedanib, pirfenidone and mycophenolic acid in the range of 2.12-2.87 μm and FPF (of recovery dose) of nintedanib, pirfenidone and mycophenolic acid in the range of 56.24-60.13%. T10 and T37 showed higher drug deposition at NGI stage 6 to MOC. Furthermore, T10, T36 and T37 showed similar drug distribution of nintedanib, pirfenidone and mycophenolic acid through NGI stages (FIG. 22). However, the increase in % drug loading (T38) decreased aerodynamic performance of pirfenidone (FPF, 50.70% and MMAD, 2.79 μm), while higher drug loading did not affect aerodynamic performance of nintedanib (FPF, 58.67% and MMAD, 2.37 μm) and mycophenolic acid (FPF, 59.21% and MMAD, 2.45 μm) compared with T36. In T38, the higher drug deposition of pirfenidone at the throat induction port was shown compared with T36 (FIG. 22). Moreover, T40 containing fumaric acid and lactose showed lower FPF (of recovery dose) of nintedanib, pirfenidone and mycophenolic acid were 53.77%, 41.27% and 55.97%, respectively compared with T36 containing lactose (Table 14). In drug distribution, T40 showed higher drug deposition of each drug at the device, mount piece and throat induction port compared with T37 (FIG. 23).

Aerodynamic performance of NM08 showed MMAD of nintedanib at 1.28 μm and mycophenolic acid at 1.31 μm. FPF (of recovery dose) of nintedanib and mycophenolic acid were 76.33% and 74.19%, respectively. In NM09, MMAD of nintedanib was 1.12 μm and mycophenolic acid was 1.45 μm. FPF (of recovery dose) of nintedanib and mycophenolic acid were 81.95% and 78.11%, respectively (Table 14). Furthermore, NM08 and NM09 show similar drug deposition of each drug through NGI stages. NM09 showed higher drug deposition of each drug at NGI stage 4-7 compared with NM08 (FIG. 24).

TABLE 14
Aerodynamic performance of each formulation

| | % FPF (of recovered dose) | | | % FPF (of delivered dose) | | | MMAD (μm) | | | % EF (of loaded dose) | | |
|---|---|---|---|---|---|---|---|---|---|---|---|---|
| Formulations | NIN | PIR | MA | NIN | PIR | MA | NIN | PIR | MA | NIN | PIR | MA |
| T10 | 86.54 | 84.28 | 86.43 | 90.47 | 89.13 | 90.38 | 0.80 | 0.81 | 0.80 | 95.66 | 94.56 | 98.71 |
| T11 | 88.62 | 54.59 | 87.00 | 92.64 | 61.63 | 91.63 | 0.70 | 0.93 | 0.74 | 95.66 | 88.58 | 94.95 |
| T35 | 57.99 | 58.33 | 54.93 | 62.11 | 63.47 | 59.85 | 2.12 | 2.87 | 2.18 | 93.40 | 85.94 | 91.77 |
| T36 | 57.81 | 56.24 | 60.13 | 61.61 | 62.13 | 64.60 | 2.74 | 2.62 | 2.42 | 93.80 | 90.44 | 93.08 |
| T37 | 87.32 | 80.35 | 87.12 | 90.14 | 86.55 | 90.84 | 0.82 | 0.83 | 0.84 | 96.87 | 92.83 | 79.88 |
| T38 | 52.81 | 50.70 | 55.11 | 85.82 | 55.35 | 58.81 | 2.37 | 2.79 | 2.55 | 74.35 | 91.58 | 93.70 |
| T39 | 58.67 | 50.70 | 59.21 | 62.32 | 55.35 | 63.88 | 2.37 | 2.79 | 2.45 | 94.12 | 91.58 | 92.66 |
| T40 | 53.77 | 41.27 | 55.97 | 61.03 | 46.49 | 61.91 | 2.22 | 3.14 | 2.26 | 90.26 | 83.30 | 90.29 |
| NM08 | 76.33 | — | 74.19 | 78.79 | — | 77.58 | 1.28 | — | 1.31 | 96.88 | — | 95.63 |
| NM09 | 81.95 | — | 78.11 | 85.90 | — | 83.33 | 1.12 | — | 1.45 | 93.74 | — | 95.40 |

### Example 6—Inhaled Pirfenidone Compositions, Characteristics and Aerodynamic Properties

Materials. Pirfenidone was purchased from Oakwood Products Inc.

Dry Powder Inhaler Preparations. Dry powders for inhalation were prepared by thin-film freezing (TFF) technique. Pirfenidone was dissolved in acetonitrile and sonicated for 10 minutes to obtain a clear solution. Stabilizers and other excipients were separately dissolved in water and sonicated for 10 minutes to obtain the clear solution. Afterward, aliquot the drug solution and excipient solutions into a bottle and then add water and acetonitrile to obtain total volume in acetonitrile and water mixture. Distearoylphosphatidylcholine (DSPC) was added into formulations and sonicated for 20 minutes. The compositions of each formulation were showed in Table 15. Approximately 15 microlites of each formulation was dropped at 10 cm height onto a rotating drum that was stainless steel cooled by cryogenic. Liquid nitrogen was used to control temperature through TFF process at −100° C. to −120° C. The frozen disks of samples were collected into a stainless-steel chamber filled with liquid nitrogen and preserved in a −80° C. freezer before transferring to a lyophilizer. A lyophilizer was used to dry frozen samples by removing the solvents. The samples were primary dried at −40° C. for 20 hours, then ramped to 25° C. over 20 hours and secondary dried at 25° C. for 20 hours for drying process. Pressure during the drying process was controlled at 100 mTorr.

TABLE 15
Compositions of each formulation.

| | | | | | Solid | |
|---|---|---|---|---|---|---|
| Formu- | | % | | % | content | |
| lations | Drug | w/w | Excipient | w/w | (% w/v) | Diluent |
| P09 | Pirfenidone | 25.00 | Leucine | 25.00 | 0.50 | Water:ACN |
| | | | Captisol ® | 50.00 | | (30:70) |
| P17 | Pirfenidone | 10.00 | Leucine | 38.00 | 0.25 | Water:ACN |
| | | | Captisol ® | 50.00 | | (30:70) |
| | | | PVP K25 | 2.00 | | |
| P18 | Pirfenidone | 10.00 | Leucine | 35.00 | 0.25 | Water:ACN |
| | | | Captisol ® | 50.00 | | (30:70) |
| | | | DSPC | 5.00 | | |
| P20 | Pirfenidone | 10.00 | Lactose | 23.00 | 0.25 | Water:ACN |
| | | | Leucine | 65.00 | | (50:50) |
| | | | PVP K25 | 2.00 | | |
| P21 | Pirfenidone | 10.00 | Lactose | 20.00 | 0.25 | Water:ACN |
| | | | Leucine | 65.00 | | (50:50) |
| | | | DSPC | 5.00 | | |
| P22 | Pirfenidone | 10.00 | Leucine | 40.00 | 0.25 | Water:ACN |
| | | | Captisol ® | 50.00 | | (30:70) |
| P23 | Pirfenidone | 10.00 | Leucine | 70.00 | 0.25 | Water:ACN |
| | | | Captisol ® | 20.00 | | (50:50) |
| P24 | Pirfenidone | 15.00 | Lactose | 20.00 | 0.50 | Water:ACN |
| | | | Leucine | 65.00 | | (50:50) |
| P25 | Pirfenidone | 15.00 | Leucine | 35.00 | 0.25 | Water:ACN |
| | | | Captisol ® | 50.00 | | (30:70) |
| P26 | Pirfenidone | 15.00 | Leucine | 33.00 | 0.25 | Water:ACN |
| | | | Captisol ® | 50.00 | | (30:70) |
| | | | PVP K25 | 2.00 | | |
| P27 | Pirfenidone | 15.00 | Leucine | 30.00 | 0.25 | Water:ACN |
| | | | Captisol ® | 50.00 | | (30:70) |
| | | | DSPC | 5.00 | | |

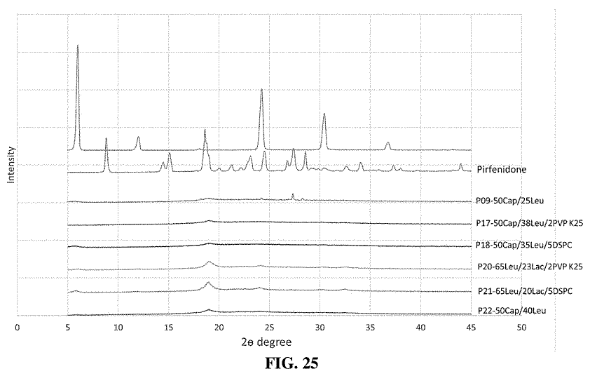

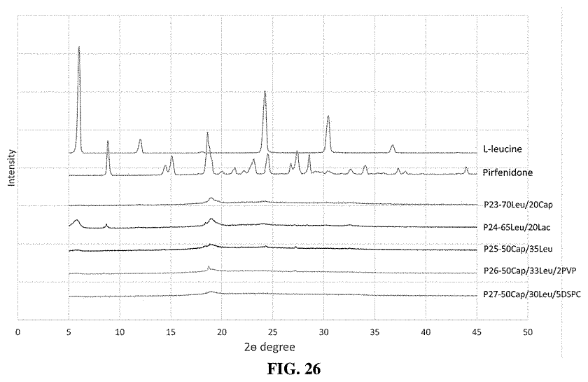

Physicochemical Properties. The amorphous morphology of TFF powders was investigated by X-ray Powder Diffraction (XRPD). X-ray diffraction (MiniFlex 600, Rigaku Co., Tokyo, Japan) measure from 5° to 45° over a 2θ range (0.04° step, 2°/min, 40 kV, 15 mA). Inhaled pirfenidone formulations including P17, P18, P22 and P27 showed amorphous morphology of pirfenidone and excipients. However, P4, P20, P23, P25 and P26 showed peaks of pirfenidone that presented crystalline morphology. P21 and P24 showed peaks of pirfenidone and leucine that presented crystalline morphology (FIG. 25-26).

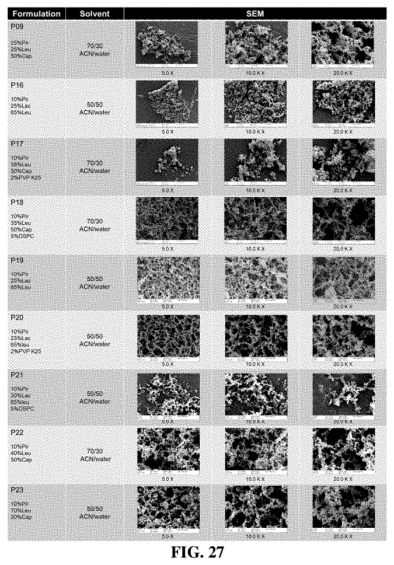

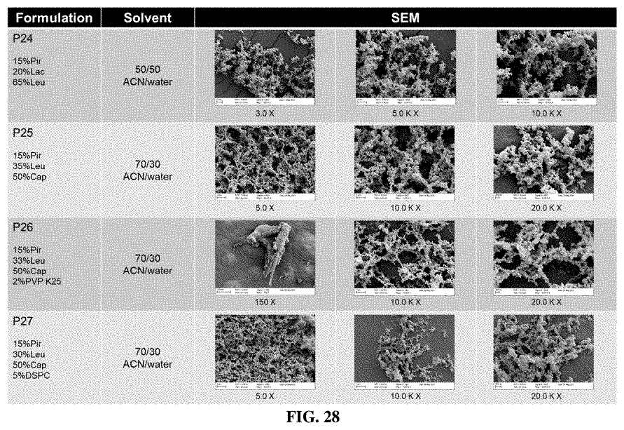

The surface morphology of TFF powders was investigated by Scanning Electron Microscopy (SEM) (Zeiss Supra 40 V SEM, Carl Zeiss, Heidenheim an der Brenz, Germany). TFF powders were placed onto carbon tape and sputter coated with 15 mm thickness of platinum/palladium alloy (Pt/Pd) before capturing the images. In inhaled pirfenidone formulations prepared, P16-P25 and P27-P28 showed brittle matrix and homogenous TFF powders. Primary particles size was shown below 1 μm (FIG. 27-28). Thus, TFF powders containing pirfenidone 10% w/w were modified as brittle matrix structure even prepared by excipients and solvent system. However, P26 containing 15% w/w pirfenidone showed large rod-shape particles of pirfenidone (over 100 μm) mixed with brittle matrix powders (FIG. 28). Therefore, high drug loading of pirfenidone can cause phase separation of inhaled pirfenidone powders.

Aerodynamic particle size distribution. Aerodynamic particle size distribution was investigated by a Next Generation Pharmaceutical Impactor (NGI) (MSP Co. Shoreview, Minn.) connected with High Capacity Pump (model HCP5, Copley Scientific, Nottingham, UK), and Critical Flow Controller (model TPK 2000, Copley Scientific, Nottingham, UK) was used to control air flow rate. A HPMC capsule size 3 containing TFF powders (approximately 5 mg per capsules), was loaded into high resistant RS00 dry powder inhaler (Plastiape, Osnago, Italy). TFF powders (5 mg) were delivered into the NGI through the USP induction port, stages 1-7, and micro-orifice collector (MOC) at the flow rate of 60 L/min for 4 s per each actuation. The deposited powders from the capsule, inhaler (device), adapter (mouthpiece), induction port (throat), stages 1-7, and the micro-orifice collector (MOC) were collected by diluting with a mixture of acetonitrile and water (50/50% v/v).

The drug content was analyzed with a Thermo Scientific™ Dionex™ UltiMate™ 3000 HPLC system (Thermo Scientific, Sunnyvale, Calif., USA) with a Waters Xbridge C18 column (4.6×150 mm, 3.5 μm) (Milford, Mass.). A gradient method that was 25% of acetonitrile to 6 minutes, 60% of acetonitrile to 8 minutes, and 25% of Acetonitrile to 9 minutes was also used to detect the content of pirfenidone. The retention time of pirfenidone was 4.53 minutes.

In the results of aerodynamic properties, P09 containing 25% w/w of pirfenidone showed the highest MMAD at 4.03 μm. In term of 10% w/w pirfenidone formulations, P20 containing 65% w/w leucine, 23% w/w lactose and 2% w/w PVP K25 showed the lowest MMAD at 1.14 μm, while P18 containing 50% w/w Captisol®, 35% w/w leucine and 5% w/w DSPC showed the higher MMAD at 2.36 μm. P17, P22 and P23 containing lactose and leucine showed MMAD at 2.08, 2.13 and 1.48 μm respectively, while P21 containing leucine and Captisol® showed MMAD at 1.31 μm. In term of 15% w/w pirfenidone formulations, P24 containing 65% w/w leucine and 20% w/w lactose showed MMAD at 2.55 μm, while P25 containing 50% w/w Captisol® and 35% w/w leucine showed the higher MMAD at 4.00 μm. P26 and P27 showed MMAD at 3.29 and 3.42 μm respectively (Table. 16).

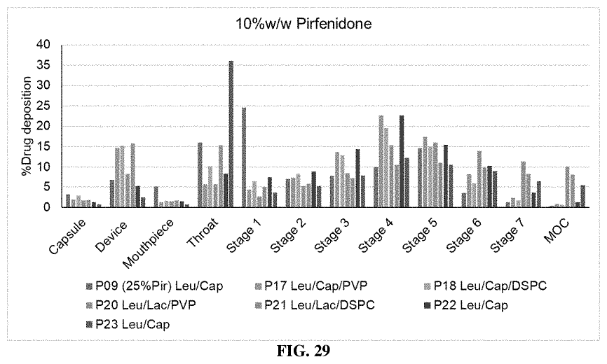

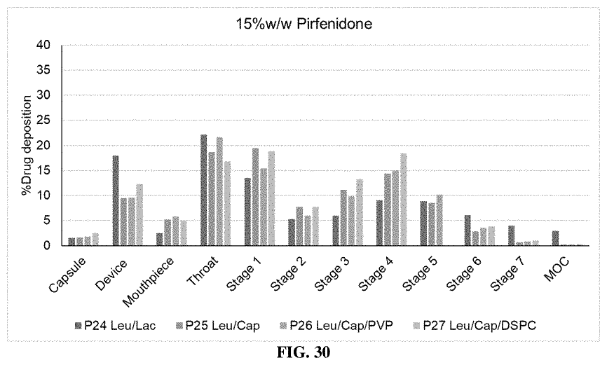

In FPF results of recovered dose, P09 containing 25% w/w showed the low FPF at 38.76% compared with inhaled pirfenidone formulations containing 10% w/w of pirfenidone. P20 containing 65% w/w leucine, 23% w/w lactose and 2% w/w PVP K25 showed the highest FPF at 76.53%. P17, P18, P19, P21, P22 and P23 showed high FPF in the range of 52.58-69.62%. In FPF results of delivered dose, P09 containing 25% w/w pirfenidone showed the lowest FPF at 43.04% compared with P20 containing 10% w/w pirfenidone that showed the highest FPF at 84.96%. P17, P18, P19, P21, P22 and P23 showed high FPF in the range of 54.29-74.43% (Table 16). In addition, P20 showed higher drug deposition at NGI stage 6-8 and MOC, while P23 showed the highest deposition at the throat induction port (FIG. 29).

In terms of 15% w/w pirfenidone formulations, inhaled pirfenidones formulations showed lower FPF (of recovered dose) compared with 10% w/w pirfenidone. P24 containing leucine and lactose showed the lowest FPF of recovered dose at 38.16%, while P25 containing leucine and Captisol® showed the low FPF at 39.40%. P26 and P27 containing PVP K25 and DSPC showed FPF at 40.89 and 44.69% respectively. In FPF results of delivered dose, P24 containing leucine and lactose showed the low FPF at 47.40%, while P25 containing leucine and Captisol® showed the low FPF at 44.32%. P26 and P27 containing PVP K25 and DSPC showed FPF at 46.15 and 51.50% respectively (Table 16). In addition, P27 containing leucine, Captisol® and DSPC showed higher drug deposition at NGI stage 2-5 compared with P24-P26 (FIG. 30). However, 15% w/w pirfenidone formulations showed higher drug deposition at device to NGI stage 1 compared with P16-P23 (10% w/w pirfenidone). N15 and N17 containing lactose showed different drug deposition compared to the initial point (FIG. 29-30).

TABLE 16
Aerodynamic performance of inhaled pirfenidone.

| | % FPF | % FPF | | % EF | % |
|---|---|---|---|---|---|
| | (of | (of | | (of | Recovery |
| | recovered | delivered | MMAD | recovered | (of loaded |
| Formulation | dose) | dose) | (μm) | dose) | dose) |
| P09 | 38.76 | 43.04 | 4.03 | 90.06 | 98.30 |
| P17 | 66.64 | 79.92 | 2.08 | 83.39 | 86.54 |
| P18 | 57.49 | 69.98 | 2.36 | 81.92 | 79.22 |
| P20 | 76.53 | 84.96 | 1.14 | 90.07 | 80.62 |
| P21 | 56.08 | 68.01 | 1.31 | 82.45 | 77.51 |
| P22 | 69.62 | 74.43 | 2.13 | 93.54 | 71.97 |
| P23 | 52.58 | 54.29 | 1.48 | 96.85 | 82.41 |
| P24 | 38.16 | 47.40 | 2.55 | 80.51 | 77.01 |
| P25 | 39.40 | 44.32 | 4.00 | 88.91 | 85.05 |
| P26 | 40.89 | 46.15 | 3.29 | 88.59 | 84.39 |
| P27 | 44.69 | 51.50 | 3.42 | 86.77 | 85.27 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
U.S. Pat. No. 9,517,204
PCT Publication No. WO 2012/106382
PCT Publication No. WO 2018/108669
“American Thoracic Society/European Respiratory Society International Multidisciplinary Consensus Classification of the Idiopathic Interstitial Pneumonias”, Am. J. Respir. Crit. Care Med., 165(2):277-304, 2002.
“Summary of Product Characteristics: Esbriet 267 mg hard capsules”, Germany: Roche Registration, 2015.
“Summary of Product Characteristics: Ofev”, Germany: Boehringer Ingelheim International GmbH, 2019.
American Lung Association, “Interstitial Lung Disease (ILD)” [Internet]. Available from: https://www.lung.org/lung-health-and-diseases/lung-disease-lookup/interstitial-lung-disease/. Cited Jan. 25, 2020.
Demedts et al., N. Engl. J. Med., 353(21):2229-2242, 2005.
Didiasova et al., FASEB J., 31(5):1916-1928, 2017.
Dugas et al., Int. J. Pharm., 441(1):19-29, 2013.
Flaherty et al., N. Engl. J. Med., 381(18):1718-1727, 2019.
Flaherty et al., Eur. Respir. J., 52(2):1800230, 2018.
Fujiyama et al., Xenobiotica, 39(5):407-414, 2009.
Hickey and Mansour, New York, N.Y.: CRC Press, p. 303-305, 2019.
Hiwarkar et al., Clin. Transplant., 25(2):222-227, 2011.
Inomata et al., Respir Res., 15(16):1-14, 2014.
Jonsson and Carlsten, Cell. Immunol., 216(1):93-101, 2002.
Khoo et al., J. Aerosol Med. Pulmon. Drug Deliv., 33(1):15-20, 2020.
Lopez-de la Mora et al., Int. J. Med. Sci., 12(11):840-847, 2015.
Marathe and Schuck, Clin. Pharm. Rev., 1-140, 2014.
Margaritopoulos et al., Core Evid., 11:11-22, 2016.
Moon et al., Mol. Pharm., 16(5):1799-1812, 2019.
Morath et al., Clin. Transplant., 20(s17):25-29, 2006.
Nambiar et al., PLoS One, 12(4):e0176312, 2017.
Overhoff et al., J. Drug Deliv. Sci. Technol., 19(2):89-98, 2009.
Raghu et al., Am. J. Respir. Crit. Care Med., 192(2):e3-e19, 2015.
Richeldi et al., N. Engl. J. Med., 370(22):2071-2082, 2014.
Schraufnagel, Am. Thoracic Soc., p. 99-103, 2010.
Trulock et al., J. Heart Lung Transplant., 26(8):782-795, 2007.
Wilson and Raghu, Eur. Respir. J., 46(4):883, 2015.

What is claimed is:

1. A pharmaceutical composition comprising particles, wherein individual particles of the composition comprise a combination of two or more active pharmaceutical ingredients selected from:
(A) nintedanib;
(B) pirfenidone; and/or
(C) mycophenolic acid.

2. The pharmaceutical composition of claim 1, wherein the pharmaceutical composition is formulated for administration via inhalation.

3. The pharmaceutical composition of either claim 1 or claim 2, wherein the particles comprise nintedanib and pirfenidone.

4. The pharmaceutical composition of either claim 1 or claim 2, wherein the particles comprise nintedanib and mycophenolic acid.

5. The pharmaceutical composition according to any one of claims 1-4, wherein the particles comprise nintedanib, pirfenidone, and mycophenolic acid.

6. The pharmaceutical composition according to any one of claims 1-5, wherein the particles further comprise an excipient.

7. The pharmaceutical composition according to any one of claims 1-6, wherein the excipient is a sugar or sugar alcohol.

8. The pharmaceutical composition according to any one of claims 1-7, wherein the excipient is a sugar.

9. The pharmaceutical composition of claim 8, wherein the sugar is lactose, sucrose, and trehalose.

10. The pharmaceutical composition according to anyone of claims 1-7, wherein the excipient is a sugar alcohol.

11. The pharmaceutical composition of claim 10, wherein the sugar alcohol is mannitol.

12. The pharmaceutical composition according to any one of claims 1-6, wherein the excipient is an acid.

13. The pharmaceutical composition of claim 12, wherein the acid is a carboxylic acid.

14. The pharmaceutical composition of either claim 12 or claim 13, wherein the acid is fumaric acid.

15. The pharmaceutical composition according to any one of claims 1-6, wherein the excipient is a cyclodextrin.

16. The pharmaceutical composition of claim 15, wherein the excipient is a sulfobutyl ether β-cyclodextrin.

17. The pharmaceutical composition according to any one of claims 1-6, wherein the excipient is an amino acid.

18. The pharmaceutical composition of claim 17, wherein the amino acid is a hydrophobic amino acid.

19. The pharmaceutical composition of claim 18, wherein the amino acid is leucine.

20. The pharmaceutical composition according to any one of claims 1-6, wherein the excipient is a flow enhancing agent.

21. The pharmaceutical composition of claim 20, wherein the flow enhancing agent is magnesium stearate.

22. The pharmaceutical composition according to any one of claims 1-6, wherein the excipient is lecithin.

23. The pharmaceutical composition according to any one of claims 1-6, wherein the excipient is a pharmaceutically acceptable polymer.

24. The pharmaceutical composition of claim 23, wherein the pharmaceutically acceptable polymer is a non-cellulosic polymer.

25. The pharmaceutical composition of claim 24, wherein the non-cellulosic polymer is a non-ionizable non-cellulosic polymer.

26. The pharmaceutical composition according to any one of claims 23-25, wherein the pharmaceutical acceptable polymer is polyvinylpyrrolidone.

27. The pharmaceutical composition of claim 26, wherein the polyvinylpyrrolidone comprises a molecular weight from about 10,000 to about 40,000.

28. The pharmaceutical composition of claim 27, wherein the molecular weight is from about 20,000 to about 30,000.

29. The pharmaceutical composition of claim 27 or claim 28, wherein the molecular weight is about 24,000.

30. The pharmaceutical composition according to any one of claims 1-6, wherein the excipient is a cyclodextrin.

31. The pharmaceutical composition of claim 30, wherein the cyclodextrin is a β-cyclodextrin.

32. The pharmaceutical composition of claim 31, wherein the cyclodextrin is modified with one or more sulfonyl groups.

33. The pharmaceutical composition of claim 32, wherein the cyclodextrin is substituted with 6.5 units of sulfobutylether.

34. The pharmaceutical composition of claim 33, wherein the cyclodextrin is 6.5-sulfobutylether-β-cyclodextrin.

35. The pharmaceutical composition according to any one of claims 1-29, wherein the particles comprise from about 10% w/w to about 80% w/w of the active pharmaceutical ingredients.

36. The pharmaceutical composition according to any one of claims 1-35, wherein the particles comprise from about 15% w/w to about 60% w/w of the active pharmaceutical ingredients.

37. The pharmaceutical composition according to any one of claims 1-36, wherein the particles comprise from about 20% w/w to about 40% w/w of the active pharmaceutical ingredients.

38. The pharmaceutical composition according to any one of claims 1-37, wherein the particles comprise about 25% w/w of the active pharmaceutical ingredients.

39. The pharmaceutical composition according to any one of claims 1-38, wherein the particles comprise a weight ratio of nintedanib and pirfenidone from about 5:1 to about 1:10.

40. The pharmaceutical composition of claim 39, wherein the weight ratio of nintedanib and pirfenidone in the particles is from about 1:1 to about 1:5.

41. The pharmaceutical composition of claim 40, wherein the weight ratio of nintedanib and pirfenidone in the particles is about 1:3.

42. The pharmaceutical composition according to any one of claims 1-41, wherein the particles comprise a weight ratio of nintedanib and mycophenolic acid from about 5:1 to about 1:10.

43. The pharmaceutical composition of claim 42, wherein the weight ratio of nintedanib and mycophenolic acid in the particles is from about 1:1 to about 1:5.

44. The pharmaceutical composition of claim 43, wherein the weight ratio of nintedanib and mycophenolic acid in the particles is about 1:3.

45. The pharmaceutical composition according to any one of claims 1-44, wherein the particles comprise a weight ratio of pirfenidone and mycophenolic acid from about 10:1 to about 1:10.

46. The pharmaceutical composition of claim 45, wherein the weight ratio of pirfenidone and mycophenolic acid in the particles is from about 5:1 to about 1:5.

47. The pharmaceutical composition of claim 46, wherein the weight ratio of pirfenidone and mycophenolic acid in the particles is about 1:1.

48. The pharmaceutical composition according to any one of claims 6-47, wherein the particles comprise from about 50% w/w to about 95% w/w of the excipient.

49. The pharmaceutical composition according to any one of claims 6-48, wherein the particles comprise from about 65% w/w to about 85% w/w of the excipient.

50. The pharmaceutical composition according to any one of claims 6-49, wherein the particles comprise about 75% w/w of the excipient.

51. The pharmaceutical composition according to any one of claims 1-50, wherein the particles comprise at least 80% of one or more of the active pharmaceutical ingredients in the amorphous phase.

52. The pharmaceutical composition of claim 51, wherein at least 90% of one or more of the active pharmaceutical ingredients is in the amorphous phase.

53. The pharmaceutical composition of claim 52, wherein at least 95% of one or more of the active pharmaceutical ingredients is in the amorphous phase.

54. The pharmaceutical composition of claim 53, wherein at least 98% of one or more of the active pharmaceutical ingredients is in the amorphous phase.

55. The pharmaceutical composition of claim 54, wherein at least 99% of one or more of the active pharmaceutical ingredients is in the amorphous phase.

56. The pharmaceutical composition according to any one of claims 51-55, wherein the active pharmaceutical ingredient in the amorphous form is nintedanib.

57. The pharmaceutical composition according to any one of claims 51-56, wherein the active pharmaceutical ingredient in the amorphous form is pirfenidone.

58. The pharmaceutical composition according to any one of claims 51-57, wherein the active pharmaceutical ingredient in the amorphous form is mycophenolic acid.

59. The pharmaceutical composition according to any one of claims 51-58, wherein the active pharmaceutical ingredient in the amorphous form is nintedanib and pirfenidone.

60. The pharmaceutical composition according to any one of claims 51-58, wherein the active pharmaceutical ingredient in the amorphous form is nintedanib and mycophenolic acid.

61. The pharmaceutical composition according to any one of claims 51-58, wherein the active pharmaceutical ingredient in the amorphous form is nintedanib, pirfenidone, and mycophenolic acid.

62. The pharmaceutical composition according to any one of claims 6-61, wherein the particles comprise at least 80% of the excipient in the amorphous phase.

63. The pharmaceutical composition according to any one of claims 6-62, wherein at least 90% of excipient is in the amorphous phase.

64. The pharmaceutical composition according to any one of claims 6-63, wherein at least 95% of the excipient is in the amorphous phase.

65. The pharmaceutical composition according to any one of claims 6-64, wherein at least 98% of the excipient is in the amorphous phase.

66. The pharmaceutical composition according to any one of claims 6-65, wherein at least 99% of the excipient is in the amorphous phase.

67. The pharmaceutical composition according to any one of claims 6-62, wherein the particles comprise at least 80% of the excipient in the crystalline phase.

68. The pharmaceutical composition according to any one of claims 6-62 and 67, wherein at least 90% of excipient is in the crystalline phase.

69. The pharmaceutical composition according to any one of claims 6-62, 67, and 68, wherein at least 95% of the excipient is in the crystalline phase.

70. The pharmaceutical composition according to any one of claims 6-62 and 67-69, wherein at least 98% of the excipient is in the crystalline phase.

71. The pharmaceutical composition according to any one of claims 6-62 and 67-70, wherein at least 99% of the excipient is in the crystalline phase.

72. The pharmaceutical composition according to any one of claims 1-71, wherein the particles comprise a matrix structure.

73. The pharmaceutical composition according to any one of claims 1-72, wherein the particles comprise a homogenous mixture of the active pharmaceutical ingredients.

74. The pharmaceutical composition according to any one of claims 1-73, wherein the particles containing nintedanib has a mass median aerodynamic diameter from about 1.0 μm to about 6.0 μm.

75. The pharmaceutical composition of claim 74, wherein the mass median aerodynamic diameter of the particles containing nintedanib is from about 2.0 μm to about 5.0 μm.

76. The pharmaceutical composition of claim 75, wherein the mass median aerodynamic diameter of the particles containing nintedanib is from about 2.5 μm to about 4.5 μm.

77. The pharmaceutical composition according to any one of claims 1-76, wherein the particles containing pirfenidone has a mass median aerodynamic diameter from about 1.0 μm to about 7.0 μm.

78. The pharmaceutical composition of claim 77, wherein the mass median aerodynamic diameter of the particles containing pirfenidone is from about 2.0 μm to about 6.0 μm.

79. The pharmaceutical composition of claim 78, wherein the mass median aerodynamic diameter of the particles containing pirfenidone is from about 3.0 μm to about 5.0 μm.

80. The pharmaceutical composition according to any one of claims 1-79, wherein the particles containing mycophenolic acid has a mass median aerodynamic diameter from about 1.0 μm to about 6.0 μm.

81. The pharmaceutical composition of claim 80, wherein the mass median aerodynamic diameter of the particles containing mycophenolic acid is from about 1.5 μm to about 5.0 μm.

82. The pharmaceutical composition of claim 81, wherein the mass median aerodynamic diameter of the particles containing mycophenolic acid is from about 2.0 μm to about 4.5 μm.

83. The pharmaceutical composition according to any one of claims 1-82, wherein the particles containing nintedanib has a geometric standard deviation (GSD) from about 1 to about 7.5.

84. The pharmaceutical composition of claim 83, wherein the geometric standard deviation of the particles containing nintedanib is from about 1.5 to about 5.

85. The pharmaceutical composition of claim 84, wherein the geometric standard deviation of the particles containing nintedanib is from about 2 to about 4.

86. The pharmaceutical composition according to any one of claims 1-85, wherein the particles containing pirfenidone has a geometric standard deviation (GSD) from about 1 to about 8.

87. The pharmaceutical composition of claim 86, wherein the geometric standard deviation of the particles containing pirfenidone is from about 1.5 to about 6.5.

88. The pharmaceutical composition of claim 87, wherein the geometric standard deviation of the particles containing pirfenidone is from about 2 to about 5.5.

89. The pharmaceutical composition according to any one of claims 1-88, wherein the particles containing mycophenolic acid has a geometric standard deviation (GSD) from about 1 to about 7.5.

90. The pharmaceutical composition of claim 89, wherein the geometric standard deviation of the particles containing mycophenolic acid is from about 1.5 to about 5.

91. The pharmaceutical composition of claim 90, wherein the geometric standard deviation of the particles containing mycophenolic acid is from about 2 to about 4.

92. The pharmaceutical composition according to any one of claims 1-91, wherein the pharmaceutical composition has a fine particle fraction of recovered dose of the particles containing nintedanib is greater than 15%.

93. The pharmaceutical composition of claim 92, wherein the fine particle fraction of recovered dose of the particles containing nintedanib is greater than 20%.

94. The pharmaceutical composition of claim 93, wherein the fine particle fraction of recovered dose of the particles containing nintedanib is greater than 25%.

95. The pharmaceutical composition according to any one of claims 1-94, wherein the pharmaceutical composition has a fine particle fraction of recovered dose of the particles containing pirfenidone is greater than 15%.

96. The pharmaceutical composition of claim 95, wherein the fine particle fraction of recovered dose of the particles containing pirfenidone is greater than 20%.

97. The pharmaceutical composition of claim 96, wherein the fine particle fraction of recovered dose of the particles containing pirfenidone is greater than 25%.

98. The pharmaceutical composition according to any one of claims 1-97, wherein the pharmaceutical composition has a fine particle fraction of recovered dose of the particles containing mycophenolic acid is greater than 15%.

99. The pharmaceutical composition of claim 98, wherein the fine particle fraction of recovered dose of the particles containing mycophenolic acid is greater than 18%.

100. The pharmaceutical composition of claim 99, wherein the fine particle fraction of recovered dose of the particles containing mycophenolic acid is greater than 20%.

101. The pharmaceutical composition according to any one of claims 1-100, wherein the pharmaceutical composition has a fine particle fraction of delivered dose of the particles containing nintedanib is greater than 25%.

102. The pharmaceutical composition of claim 101, wherein the fine particle fraction of delivered dose of the particles containing nintedanib is greater than 30%.

103. The pharmaceutical composition of claim 102, wherein the fine particle fraction of delivered dose of the particles containing nintedanib is greater than 35%.

104. The pharmaceutical composition according to any one of claims 1-104, wherein the pharmaceutical composition has a fine particle fraction of delivered dose of the particles containing pirfenidone is greater than 20%.

105. The pharmaceutical composition of claim 104, wherein the fine particle fraction of delivered dose of the particles containing pirfenidone is greater than 25%.

106. The pharmaceutical composition of claim 105, wherein the fine particle fraction of delivered dose of the particles containing pirfenidone is greater than 30%.

107. The pharmaceutical composition according to any one of claims 1-106, wherein the pharmaceutical composition has a fine particle fraction of delivered dose of the particles containing mycophenolic acid is greater than 20%.

108. The pharmaceutical composition of claim 107, wherein the fine particle fraction of delivered dose of the particles containing mycophenolic acid is greater than 25%.

109. The pharmaceutical composition of claim 108, wherein the fine particle fraction of delivered dose of the particles containing mycophenolic acid is greater than 30%.

110. The pharmaceutical composition according to any one of claims 1-109, wherein the pharmaceutical composition has a percentage recovery as a function of the loaded dose of the particles containing nintedanib is greater than 60%.

111. The pharmaceutical composition of claim 110, wherein the percentage recovery as a function of the loaded dose of the particles containing nintedanib is greater than 65%.

112. The pharmaceutical composition of claim 111, wherein the percentage recovery as a function of the loaded dose of the particles containing nintedanib is greater than 70%.

113. The pharmaceutical composition according to any one of claims 1-112, wherein the pharmaceutical composition has a percentage recovery as a function of the loaded dose of the particles containing pirfenidone is greater than 60%.

114. The pharmaceutical composition of claim 113, wherein the percentage recovery as a function of the loaded dose of the particles containing pirfenidone is greater than 65%.

115. The pharmaceutical composition of claim 114, wherein the percentage recovery as a function of the loaded dose of the particles containing pirfenidone is greater than 70%.

116. The pharmaceutical composition according to any one of claims 1-115, wherein the pharmaceutical composition has a percentage recovery as a function of the loaded dose of the particles containing mycophenolic acid is greater than 70%.

117. The pharmaceutical composition of claim 116, wherein the percentage recovery of the loaded dose as a function of the particles containing mycophenolic acid is greater than 75%.

118. The pharmaceutical composition of claim 117, wherein the percentage recovery of the loaded dose as a function of the particles containing mycophenolic acid is greater than 80%.

119. The pharmaceutical composition according to any one of claims 1-118, wherein the pharmaceutical composition has an emitted fraction of the particles containing nintedanib is greater than 60% as measured using a NGI.

120. The pharmaceutical composition of claim 119, wherein the emitted fraction of the particles containing nintedanib is greater than 65%.

121. The pharmaceutical composition of claim 120, wherein the emitted fraction of the particles containing nintedanib is greater than 70%.

122. The pharmaceutical composition according to any one of claims 1-121, wherein the pharmaceutical composition has an emitted fraction of the particles containing pirfenidone is greater than 60% as measured using a NGI.

123. The pharmaceutical composition of claim 122, wherein the emitted fraction of the particles containing pirfenidone is greater than 65%.

124. The pharmaceutical composition of claim 123, wherein the emitted fraction of the particles containing pirfenidone is greater than 70%.

125. The pharmaceutical composition according to any one of claims 1-124, wherein the pharmaceutical composition has an emitted fraction of the particles containing mycophenolic acid is greater than 70% as measured using a NGI.

126. The pharmaceutical composition of claim 125, wherein the emitted fraction of the particles containing mycophenolic acid is greater than 75%.

127. The pharmaceutical composition of claim 126, wherein the emitted fraction of the particles containing mycophenolic acid is greater than 80%.

128. A pharmaceutical composition comprising particles, wherein individual particles of the composition comprise a combination of an active pharmaceutical ingredient and an excipient comprising:
(A) the active pharmaceutical ingredient selected from nintedanib, pirfinedone, and mycophenolic acid;
(B) the excipient;
wherein the pharmaceutical composition is formulated as a dry powder for administration via inhalation.

129. The pharmaceutical composition of claim 128, wherein the active pharmaceutical ingredient is nintedanib.

130. The pharmaceutical composition of claim 128, wherein the active pharmaceutical ingredient is pirfinedone.

131. The pharmaceutical composition of claim 128, wherein the active pharmaceutical ingredient is mycophenolic acid.

132. The pharmaceutical composition according to any one of claims 128-131, wherein the particles further comprise an excipient.

133. The pharmaceutical composition according to any one of claims 128-132, wherein the excipient is a sugar or sugar alcohol.

134. The pharmaceutical composition according to any one of claims 128-133, wherein the excipient is a sugar.

135. The pharmaceutical composition of claim 134, wherein the sugar is lactose.

136. The pharmaceutical composition according to anyone of claims 128-133, wherein the excipient is a sugar alcohol.

137. The pharmaceutical composition of claim 136, wherein the sugar alcohol is mannitol.

138. The pharmaceutical composition according to any one of claims 128-132, wherein the excipient is a cyclodextrin.

139. The pharmaceutical composition of claim 138, wherein the cyclodextrin is a β-cyclodextrin.

140. The pharmaceutical composition of claim 139, wherein the excipient is a sulfo butyl ether β-cyclodextrin.

141. The pharmaceutical composition of claim 140, wherein the cyclodextrin is 6.5-sulfobutylether-β-cyclodextrin.

142. The pharmaceutical composition according to any one of claims 128-132, wherein the excipient is an amino acid.

143. The pharmaceutical composition of claim 142, wherein the amino acid is a hydrophobic amino acid.

144. The pharmaceutical composition of claim 143, wherein the amino acid is leucine.

145. The pharmaceutical composition according to any one of claims 128-137, wherein the excipient is a flow enhancing agent.

146. The pharmaceutical composition of claim 145, wherein the flow enhancing agent is magnesium stearate or a phospholipid.

147. The pharmaceutical composition of claim 146, wherein the phospholipid is distearoylphosphatidylcholine.

148. The pharmaceutical composition according to any one of claims 128-137, wherein the excipient is lecithin.

149. The pharmaceutical composition according to any one of claims 128-137, wherein the excipient is a pharmaceutically acceptable polymer.

150. The pharmaceutical composition of claim 149, wherein the pharmaceutically acceptable polymer is a non-cellulosic polymer.

151. The pharmaceutical composition of claim 150, wherein the non-cellulosic polymer is a non-ionizable non-cellulosic polymer.

152. The pharmaceutical composition according to any one of claims 149-151, wherein the pharmaceutical acceptable polymer is polyvinylpyrrolidone.

153. The pharmaceutical composition of claim 152, wherein the polyvinylpyrrolidone comprises a molecular weight from about 10,000 to about 40,000.

154. The pharmaceutical composition of claim 153, wherein the molecular weight is from about 20,000 to about 30,000.

155. The pharmaceutical composition of claim 153 or claim 154, wherein the molecular weight is about 24,000.

156. The pharmaceutical composition according to any one of claims 128-155, wherein the particles comprise from about 10% w/w to about 80% w/w of the active pharmaceutical ingredients.

157. The pharmaceutical composition according to any one of claims 128-156, wherein the particles comprise from about 15% w/w to about 60% w/w of the active pharmaceutical ingredients.

158. The pharmaceutical composition according to any one of claims 128-157, wherein the particles comprise from about 20% w/w to about 40% w/w of the active pharmaceutical ingredients.

159. The pharmaceutical composition according to any one of claims 128-158, wherein the particles comprise about 25% w/w of the active pharmaceutical ingredients.

160. The pharmaceutical composition according to any one of claims 128-155, wherein the particles comprise from about 1% w/w to about 40% w/w of the active pharmaceutical ingredients.

161. The pharmaceutical composition according to any one of claims 128-155 and 160, wherein the particles comprise from about 5% w/w to about 20% w/w of the active pharmaceutical ingredients.

162. The pharmaceutical composition according to any one of claims 128-155, 160, and 161, wherein the particles comprise from about 7.5% w/w to about 17.5% w/w of the active pharmaceutical ingredients.

163. The pharmaceutical composition according to any one of claims 128-155 and 160-162, wherein the particles comprise about 10% w/w of the active pharmaceutical ingredients.

164. The pharmaceutical composition according to any one of claims 128-155 and 160-162, wherein the particles comprise about 15% w/w of the active pharmaceutical ingredients.

165. The pharmaceutical composition according to any one of claims 128-164, wherein the particles comprise from about 50% w/w to about 95% w/w of the excipient.

166. The pharmaceutical composition according to any one of claims 128-165, wherein the particles comprise from about 65% w/w to about 85% w/w of the excipient.

167. The pharmaceutical composition according to any one of claims 128-166, wherein the particles comprise about 75% w/w of the excipient.

168. The pharmaceutical composition according to any one of claims 128-165, wherein the particles comprise from about 75% w/w to about 95% w/w of the excipient.

169. The pharmaceutical composition according to any one of claims 128-165 and 168, wherein the particles comprise about 90% w/w of the excipient.

170. The pharmaceutical composition according to any one of claims 128-165 and 168, wherein the particles comprise about 85% w/w of the excipient.

171. The pharmaceutical composition according to any one of claims 128-167, wherein the particles comprise at least 80% of one or more of the active pharmaceutical ingredients in the amorphous phase.

172. The pharmaceutical composition of claim 171, wherein at least 90% of one or more of the active pharmaceutical ingredients is in the amorphous phase.

173. The pharmaceutical composition of claim 172, wherein at least 95% of one or more of the active pharmaceutical ingredients is in the amorphous phase.

174. The pharmaceutical composition of claim 173, wherein at least 98% of one or more of the active pharmaceutical ingredients is in the amorphous phase.

175. The pharmaceutical composition of claim 174, wherein at least 99% of one or more of the active pharmaceutical ingredients is in the amorphous phase.

176. The pharmaceutical composition according to any one of claims 128-175, wherein the particles comprise at least 80% of the excipient in the amorphous phase.

177. The pharmaceutical composition according to any one of claims 128-176, wherein at least 90% of excipient is in the amorphous phase.

178. The pharmaceutical composition according to any one of claims 128-177, wherein at least 95% of the excipient is in the amorphous phase.

179. The pharmaceutical composition according to any one of claims 128-178, wherein at least 98% of the excipient is in the amorphous phase.

180. The pharmaceutical composition according to any one of claims 128-179, wherein at least 99% of the excipient is in the amorphous phase.

181. The pharmaceutical composition according to any one of claims 128-180, wherein the particles comprise at least 80% of the excipient in the crystalline phase.

182. The pharmaceutical composition according to any one of claims 128-175 and 181, wherein at least 90% of excipient is in the crystalline phase.

183. The pharmaceutical composition according to any one of claims 128-175, 181, and 182, wherein at least 95% of the excipient is in the crystalline phase.

184. The pharmaceutical composition according to any one of claims 128-175 and 181-183, wherein at least 98% of the excipient is in the crystalline phase.

185. The pharmaceutical composition according to any one of claims 128-175 and 181-184, wherein at least 99% of the excipient is in the crystalline phase.

186. A method of preparing a pharmaceutical composition according to any one of claims 1-185 comprising:
(A) dissolving an active pharmaceutical ingredient in a solvent to obtain a pharmaceutical mixture;
(B) applying the pharmaceutical mixture to a surface at a surface temperature below 0° C. to obtain a frozen pharmaceutical mixture; and
(C) collecting the frozen pharmaceutical mixture and drying the frozen pharmaceutical mixture to obtain a pharmaceutical composition.

187. The method of claim 186, wherein the solvent is an organic solvent.

188. The method of claim 187, wherein the solvent is acetonitrile, tert-butanol, or 1,4-dioxane.

189. The method according to any one of claims 186-188 further comprising admixing the active pharmaceutical ingredient with an excipient.

190. The method according to any one of claims 186-189, wherein the pharmaceutical mixture further comprises a second solvent.

191. The method of claim 190, wherein the second solvent is water.

192. The method according to any one of claims 186-191, wherein the first solvent is mixed with the second solvent to obtain a homogenous pharmaceutical mixture.

193. The method of either claim 186 or claim 190, wherein the pharmaceutical mixture is admixed until the pharmaceutical mixture is clear.

194. The method according to any one of claims 186-193, wherein the pharmaceutical mixture comprises a solid content from about 0.05% w/v to about 5% w/v of the active pharmaceutical ingredient and the excipient.

195. The method of claim 194, wherein the solid content is from about 0.1% w/v to about 2.5% w/v of the active pharmaceutical ingredient and the excipient.

196. The method of claim 195, wherein the solid content is from about 0.15% w/v to about 1.5% w/v of the active pharmaceutical ingredient and the excipient.

197. The method of claim 196, wherein the solid content is from about 0.2% w/v to about 0.6% w/v of the active pharmaceutical ingredient and the excipient.

198. The method of claim 197, wherein the solid content is from about 0.5% w/v to about 1.25% w/v of the active pharmaceutical ingredient and the excipient.

199. The method according to any one of claims 186-198, wherein the pharmaceutical mixture is applied at a feed rate from about 0.5 mL/min to about 5 mL/min.

200. The method of claim 199, wherein the feed rate is from about 1 mL/min to about 3 mL/min.

201. The method of claim 200, wherein the feed rate is about 2 mL/min.

202. The method according to any one of claims 186-201, wherein the pharmaceutical mixture is applied with a nozzle.

203. The method of claim 202, wherein the nozzle is a needle.

204. The method according to any one of claims 186-203, wherein the pharmaceutical mixture is applied from a height from about 2 cm to about 50 cm.

205. The method of claim 204, wherein the height is from about 5 cm to about 20 cm.

206. The method of claim 205, wherein the height is about 10 cm.

207. The method according to any one of claims 186-206, wherein the surface temperature is from about 0° C. to −190° C.

208. The method of claim 207, wherein the surface temperature is from about −25° C. to about −125° C.

209. The method of claim 208, wherein the surface temperature is about −100° C.

210. The method according to any one of claims 186-209, wherein the surface is a rotating surface.

211. The method of claim 210, wherein the surface is rotating at a speed from about 5 rpm to about 500 rpm.

212. The method of claim 211, wherein the surface is rotating at a speed from about 100 rpm to about 400 rpm.

213. The method of claim 212, wherein the surface is rotating at a speed of about 200 rpm.

214. The method according to any one of claims 186-213, wherein the frozen pharmaceutical composition is dried by lyophilization.

215. The method of claim 214, wherein the frozen pharmaceutical composition is dried at a first reduced pressure.

216. The method of claim 215, wherein the first reduced pressure is from about 10 mTorr to 500 mTorr.

217. The method of claim 216, wherein the first reduced pressure is from about 50 mTorr to about 250 mTorr.

218. The method of claim 217, wherein the first reduced pressure is about 100 mTorr.

219. The method of according to any one of claims 214-218, wherein the frozen pharmaceutical composition is dried at a first reduced temperature.

220. The method of claim 219, wherein the first reduced temperature is from about 0° C. to −100° C.

221. The method of claim 220, wherein the first reduced temperature is from about −20° C. to about −60° C.

222. The method of claim 221, wherein the first reduced temperature is about −40° C.

223. The method according to any one of claims 214-222, wherein the frozen pharmaceutical composition is dried for a primary drying time period from about 3 hours to about 36 hours.

224. The method of claim 223, wherein the primary drying time period is from about 6 hours to about 24 hours.

225. The method of claim 224, wherein the primary drying time period is about 20 hours.

226. The method according to any one of claims 214-225, wherein the frozen pharmaceutical composition is dried a secondary drying time period.

227. The method of claim 226, wherein the frozen pharmaceutical composition is dried a secondary drying time at a second reduced pressure.

228. The method of claim 227, wherein the secondary drying time is at a reduced pressure is from about 10 mTorr to 500 mTorr.

229. The method of claim 228, wherein the secondary drying time is at a reduced pressure is from about 50 mTorr to about 250 mTorr.

230. The method of claim 229, wherein the secondary drying time is at a reduced pressure is about 100 mTorr.

231. The method of according to any one of claims 227-230, wherein the frozen pharmaceutical composition is dried a secondary drying time at a second reduced temperature.

232. The method of claim 231, wherein the second reduced temperature is from about 0° C. to 30° C.

233. The method of claim 232, wherein the second reduced temperature is from about 10° C. to about 30° C.

234. The method of claim 233, wherein the second reduced temperature is about 25° C.

235. The method according to any one of claims 227-234, wherein the frozen pharmaceutical composition is dried for a second time for a second time period from about 3 hours to about 36 hours.

236. The method of claim 235, wherein the second time period is from about 6 hours to about 24 hours.

237. The method of claim 236, wherein the second time period is about 20 hours.

238. The method according to any one of claims 214-237, wherein the temperature is changed from the first reduced temperature to the second reduced temperature over a ramping time period.

239. The method of claim 238, wherein the ramping time period is from about 3 hours to about 36 hours.

240. The method of claim 239, wherein the ramping time period is from about 6 hours to about 24 hours.

241. The method of claim 240, wherein the ramping time period is about 20 hours.

242. A pharmaceutical composition prepared using the methods according to any one of claims 186-241.

243. A method of treating or preventing a lung disease in a patient comprising administering to the patient in need thereof a therapeutically effective amount of a pharmaceutical composition according to any one of claims 1-185 and 242.

244. The method of claim 243, wherein the lung disease is associated with lung inflammation or fibrosis.

245. The method of either claim 243 or claim 244, wherein the lung disease is interstitial lung disease.

246. The method of claim 245, wherein the interstitial lung disease is idiopathic pulmonary fibrosis.

247. The method according to any one of claims 243-246, wherein the weight ratio of nintedanib to pirfenidone is from about 1:1 to about 1:10.

248. The method of claim 247, wherein the weight ratio is from about 1:2 to about 1:5.

249. The method of claim 248, wherein the weight ratio is from about 1:3.

250. The method according to any one of claims 243-249, wherein the weight ratio of nintedanib to mycophenolic acid is from about 1:1 to about 1:10.

251. The method of claim 250, wherein the weight ratio is from about 1:2 to about 1:5.

252. The method of claim 251, wherein the weight ratio is from about 1:3.

253. The method according to any one of claims 243-252, wherein the pharmaceutical composition comprises a dose of nintedanib is from about 1 mg/mL to about 50 mg/mL.

254. The method of claim 253, wherein the dose of nintedanib is from about 2.5 mg/mL to about 25 mg/mL.

255. The method of claim 254, wherein the dose of nintedanib is from about 5 mg/mL to about 20 mg/mL.

256. The method according to any one of claims 243-255, wherein the pharmaceutical composition comprises a dose of pirfenidone is from about 0.25 mg to about 10 mg.

257. The method of claim 256, wherein the dose of pirfenidone is from about 0.5 mg to about 7.5 mg.

258. The method of claim 257, wherein the dose of pirfenidone is from about 0.75 mg to about 5 mg.

259. The method according to any one of claims 243-258, wherein the pharmaceutical composition comprises a dose of mycophenolic acid is from about 0.25 μg/mL to about 10 μg/mL.

260. The method of claim 259, wherein the dose of mycophenolic acid is from about 0.5 μg/mL to about 7.5 μg/mL.

261. The method of claim 260, wherein the dose of mycophenolic acid is from about 0.75 μg/mL to about 5 μg/mL.

262. A method of treating or prevent reducing lung inflammation or fibrosis in a patient comprising administering to the patient in need thereof a therapeutically effective amount of a pharmaceutical composition according to any one of claims 1-185 and 242.

263. The method of claim 262, wherein the lung inflammation or fibrosis is associated with an interstitial lung disease.

264. The method of claim 263, wherein the interstitial lung disease is idiopathic pulmonary fibrosis.

265. The method according to any one of claims 243-264, wherein the pharmaceutical composition is administered once.

266. The method according to any one of claims 243-264, wherein the pharmaceutical composition is administered more than once.
